(19) United States
(12) Patent Application Publication
(10) Pub. No.: US 2012/0101038 A1
(43) Pub. Date: Apr. 26, 2012

(54) USE OF GLP-1, GLP-1 DERIVATIVES OR GLP-1 FRAGMENTS FOR SKIN REGENERATION, STIMULATION OF HAIR GROWTH, OR TREATMENT OF DIABETES

(75) Inventors: Joel F. Habener, Newton Centre, MA (US); James F. List, Princeton, NJ (US); Svetlana Mojsov, New York, NY (US)

(73) Assignee: THE GENERAL HOSPITAL CORPORATION, Boston, MA (US)

(21) Appl. No.: 13/224,371

(22) Filed: Sep. 2, 2011

Related U.S. Application Data
(63) Continuation of application No. 12/064,525, filed on Aug. 19, 2008, now abandoned.
(60) Provisional application No. 60/710,194, filed on Aug. 23, 2005.

(51) Int. Cl. A61K 38/26; A61M 37/00; A61M 5/00; A61P 17/14

(52) U.S. Cl. 514/11.7; 604/22; 604/506

(57) ABSTRACT

The present invention relates to the use of GLP-I, GLP-I derivatives or GLP-I fragments for skin regeneration or hair growth in mammals. As used for skin regeneration, GLP-I, GLP-I derivatives or GLP-I fragments can be applied to dermal wounds including burns, lacerations, cuts and scrapes. As used for hair growth, GLP-I, GLP-I derivatives or GLP-I fragments can be applied to humans suffering from alopecia, or baldness. GLP-I, GLP-I derivatives or GLP-I fragments can also be used to stimulate hair growth in animals raised for their pelts. GLP-I, GLP-I derivatives or GLP-I fragments can also be used in the redifferentiation of endothelial or skin cells into insulin producing cells, as a treatment for patients suffering from diabetes.

### BACKGROUND OF THE INVENTION

1. Field of the Invention

The present invention is directed to compositions for regenerating skin cells, promoting hair growth or treating diabetes.

2. Related Art

Dermatologists recognize many different types of hair loss, the most common by far being alopecia wherein human males begin losing scalp hair at the temples and on the crown of the head as they get older. While this type of hair loss is largely confined to males, hence its common name “male pattern baldness,” it is not unknown in women.

Efforts have been made, however, to reduce or prevent further hair loss. U.S. Published Patent Application No. 2004/0029784 A1 relates to methods of treating polycystic ovary syndrome (PCOS) comprising administering glucagon-like peptide-1 (GLP-1) to subjects suffering therefrom. The method identifies giving the subjects a therapeutically or pharmaceutically effective amount of GLP-1 required to reduce or lessen the severity of PCOS or any of its symptoms, including alopecia.

However, no known cure (i.e., stimulation of hair growth) has yet been found despite continuing attempts to discover one. Hence, there is a need for compounds or compositions which are able to stimulate hair growth.

There is also a general medical need for agents that regenerate skin cells. Such agents are useful for treating dermal wounds including burn wounds, frostbite, dermal wounds resulting from radiation damage, or open dermal wounds such as scrapes, cuts, or lacerations.

There is also a general medical need for improved methods of treating diabetes. Current treatments for diabetes include administration of insulin or stimulation of pancreatic cells with insulinotropic compounds, such as those described by U.S. Pat. No. 6,849,708, which is herein wholly incorporated by reference. One drawback of these methods, however, is the patient's dependency upon continuous and on-going administration of insulin or insulinotropic compounds. Thus, there is a general medical need for methods of treating diabetes that do not require the continuous or on-going administration to patients suffering from diabetes of insulin or insulinotropic agents.

Glucagon-like peptide-1 (GLP-1) is a peptide hormone arising by its alternative enzymatic cleavage from proglucagon, the prohormone precursor for GLP-1, which is highly expressed in enteroendocrine cells of the intestine, the alpha cells of the endocrine pancreas (islets of Langerhans), and the brain (Kieffer T. J. and Habener, J. F. Endocrin. Rev. 20:876-913 (1999); Drucker, D. J., Endocrinology 142:521-7 (2001); Holst, J. J., Diabetes Metab. Res. Rev. 18:430-41 (2002)). The initial actions of GLP-1 observed were on the insulin-producing cells of the islets, where it stimulates glucose-dependent insulin secretion. Subsequently, multiple additional antidiabetogenic actions of GLP-1 were discovered including the stimulation of the growth and inhibition of the apoptosis of pancreatic beta cells (Drucker, D. J., Endocrinology 144:5145-8 (2003); Holz, G. G. and Chepurny 0.G., Curr. Med. Chem. 10:2471-83 (2003); List, J. F. and Habener, J. F., Am. J. Physiol. Endocrinol. Metab. 286:E875-81(2004)).

The receptor for GLP-1 (GLP-1R) is expressed in a broad distribution of tissues. GLP-1R is expressed in pancreatic endocrine cells, intestinal epithelial cells, brain, lung, kidney, and heart (Kieffer T. J. and Habener, J. F., Endocrin. Rev 20:876-913 (1999); Drucker, D. J., Endocrinology 142:521-7 (2001); and Holst, J. J., Diabetes. Metab. Res. Rev. 18:430-41 (2002)), as well as in cultures of embryonic stem cells (Tuch B. et al., ISSCR 2nd Annual Meeting, Boston, Mass., 2004), and pancreatic islet-derived dedifferentiated cells (Gershengorn, M. C. et al., Science 306:2261-4 (2004)) in which GLP-1 stimulates cellular differentiation (Abraham E. J. et al., Endocrinology 143:315261 (2002)).

The GLP-1R is a seven membrane spanning, G-protein coupled receptor that activates multiple signal transduction pathways including those for cAMP/GEF, MAPIQERK, and calcium-dependent signaling. GLP-1 has been shown to exert cytoprotective and antiapoptotic actions on both pancreatic beta cells (Buteau, J. et al., Diabetologia 42:856-64 (1999)) and myocardial cells (Bose, A. K. et al., Diabetes 54:14651 (2005)), by the activation of prosurvival kinases such as P13 kinase and P42/44 MAP kinases (ERK1/2). Notably, GLP-1 has been shown to activate the epidermal growth factor (EGF) receptor, possibly via the activation of c-Src and the production of endogenous EGF-like ligands (Buteau, J. et al., Diabetes 52:124-32 (2003)).

### BRIEF SUMMARY OF THE INVENTION

Although the metabolic role of GLP-1, GLP-1 fragments or GLP-1 derivatives or their ability to influence metabolism is known, the use of these polypeptides for regenerating skin cells or stimulating hair growth has heretofore been unrecognized. The present invention is directed to using GLP-1, GLP-1 fragments or GLP-1 derivatives to regenerate skin cells or stimulate hair growth, especially hair growth on the head and preferably the top of the head or scalp.

The present invention is directed to a method of regenerating skin cells comprising administering to a mammal GLP-1, GLP-1 derivatives or GLP-1 fragments.

In one embodiment of the present invention, GLP-1, GLP-1 derivatives or GLP-1 fragments are administered to a demial wound, and preferably to the skin side of an open dermal wound. The dermal wound can be, for example, a burn wound, frostbite, or skin injury as the result of radiation damage. The open dermal wound can be, for example, a scrape, cut, or laceration. In another embodiment of the present invention, the mammal is treated with artificial skin or a bandage comprising the GLP-1, a GLP-1 fragment or a GLP-1 derivative.

The present invention is also directed to a method of stimulating hair growth comprising administering to a mammal GLP-1, a GLP-1 fragment or a GLP-1 derivative. In one embodiment of this approach, the GLP-1, GLP-1 fragment or GLP-1 derivative is applied topically.

The GLP-1, GLP-1 fragment or GLP-1 derivative can be contained in a dermal lotion, cream, cosmetic composition, patch, skin patch, bandage, surgical gauze or suture. Alternatively, the GLP-1, GLP-1 fragment or GLP-1 derivative can be administered via microneedle injections, jet injections, pressure waves, or ultrasound.

In one embodiment of the present invention, the GLP-1, GLP-1 fragment or GLP-1 derivative is administered to a human. The human can be a male or female. In one preferred embodiment, the human can be a cancer patient who is in need of skin cell regeneration or who has suffered hair loss (for example, as the result of radiation and/or chemotherapy), and is in need of stimulation of hair growth; especially on the scalp.

Alternatively, the GLP-1, GLP-1 fragment or GLP-1 derivative can be administered to an animal raised for its pelt (e.g., mink, rabbit, raccoon, fox), or to a farm animal raised for its hair (e.g., sheep, goat, alpaca), or to a domestic animal to improve its coat (e.g., cats or dogs).

The GLP-1 containing composition of the invention can also be administered prophylactically, to prevent or minimize hair loss, or to otherwise prime the hair growth response to stimulate new growth after a future loss, for example, as occurs in chemotherapy.

The present invention is also directed to kits for stimulating hair growth in a mammal comprising a container having GLP-1, a GLP-1 fragment or a GLP-1 derivative.

The present invention is also directed to a method of identifying an agent that regenerates skin cells comprising i) obtaining GLP-1R; ii) contacting a compound with the GLP-1R; and iii) determining whether the compound binds the GLP-1R; wherein the compound that binds GLP-1R is an agent that regenerates skin cells.

The present invention is also directed to a method of identifying an agent that promotes hair growth comprising i) obtaining GLP-1R; ii) contacting a compound with the GLP-1R; and iii) determining whether the compound binds the GLP-1R; wherein the compound that binds GLP-1R is an agent that promotes hair growth.

It is to be understood that both the foregoing general description and the following detailed description are exemplary and explanatory and are intended to provide further explanation of the invention as claimed.

The present invention is also directed to treating diabetes. In particular, the present invention is directed to obtaining endothelial or skin cells; culturing the cells in dedifferentiating medium, and exposing the cultured cells to GLP-1, or a GLP-1 fragment or derivative, thereby causing the cells to redifferentiate into insulin producing cells. Such cells can be administered to patients suffering from diabetes.

### BRIEF DESCRIPTION OF THE DRAWINGS/FIGURES

The accompanying drawings, which are included to provide a further understanding of the invention and are incorporated in and constitute a part of this specification, illustrate embodiments of the invention and together with the description serve to explain the principles of the invention. In the drawings:

FIG. 1A: Expression of GLP-1 receptor (GLP-1R) mRNA in mouse skin detected by RT-PCR.

FIG. 1B: Expression of GLP-1 receptor (GLP-1R) mRNA in cultured mouse skin-derived cells detected by RT-PCR.

FIG. 1C: Expression of GLP-1 receptor (GLP-1R) mRNA in cultured mouse skin-derived cells using four different PCR primer pairs.

FIG. 2: Western immunoblot of GLP-1 receptor from lysates of skin and primary skin cultures from C57B 1/6 mice.

FIG. 3A: RT-PCR of proglucagon mRNA in mouse skin.

FIG. 3B: RT-PCR of proglucagon mRNA in cultured skin-derived mouse cells.

FIG. 4A: Western blot showing ERK phosphorylation in response to GLP-1 stimulation of primary skin cell cultures in the presence of no glucose.

FIG. 4B: Western blot showing ERK phosphorylation in response to GLP-1 stimulation of primary skin cell cultures in the presence of 2.8 mM glucose.

FIG. 4C: Western blot showing ERK phosphorylation in response to GLP-1 stimulation of primary skin cell cultures in the presence of 16.7 mM glucose.

FIG. 4D: Semi-quantitative densitometric measurement of relative phosphorylation responses shown in FIG. 4A-4C.

FIG. 5: RT-PCR of nestin mRNA in cultured mouse skin-derived cells.

### DETAILED DESCRIPTION OF THE INVENTION

The present invention is directed to administering to a mammal GLP-1, GLP-1 fragments or GLP-1 derivatives to regenerate skin cells or stimulate hair growth.

In one embodiment of the present invention, GLP-1, GLP-1 derivatives or GLP-1 fragments are administered to a dermal wound, and preferably to the skin side of an open dermal wound. The dermal wound can be, for example, a burn wound, frostbite, or skin injury as the result of radiation damage. The open dermal wound can be, for example, a scrape, cut, or laceration. In another embodiment of the present invention, the mammal is treated with artificial skin or a bandage comprising the GLP-1, a GLP-1 fragment or a GLP-1 derivative.

The present invention is also directed to a method of stimulating hair growth comprising administering to a mammal GLP-1, a GLP-1 fragment or a GLP-1 derivative. In one embodiment of the present invention, the GLP-1, GLP-1 fragment or GLP-1 derivative is administered to a human. The human can be a male or female. In one preferred embodiment, the human can be a cancer patient who is in need of skin cell regeneration or who has suffered hair loss (for example, as the result of radiation and/or chemotherapy), and is in need of stimulation of hair growth; especially on the scalp. Alternatively, the human can be an individual who has suffered natural hair loss.

The GLP-1 containing composition of the invention can also be administered prophylactically, to prevent or minimize hair loss, or to otherwise prime the hair growth response to stimulate new growth after a future loss, for example, as occurs in chemotherapy.

In one embodiment of this approach, the GLP-1, GLP-1 fragment or GLP-1 derivative is applied topically, especially on the head and preferably the top of the head or scalp. The GLP-1, GLP-1 fragment or GLP-1 derivative can also be applied to other areas where additional hair growth is desired. Such areas include, for example, the pubicor groin area, the chest area, eyebrow or eyelash area, the upper lip, cheeks, chin, sideburn or other facial areas, or the armpits.

When applied topically, the GLP-1, GLP-1 fragment or GLP-1 derivative can be contained in a dermal lotion, cream, cosmetic composition, patch, skin patch, bandage, surgical gauze or suture. Alternatively, the GLP-1, GLP-1 fragment or GLP-1 derivative can be administered via intradermal injections, microneedle injections, jet injections, pressure waves, or ultrasound.

Alternatively, the GLP-1, GLP-1 fragment or GLP-1 derivative can be administered to an animal raised for its pelt, or to domestic pets or farm animals for which a thicker hair coat is desired.

The present invention is also directed to kits for stimulating hair growth in a mammal comprising a container having GLP-1, a GLP-1 fragment or a GLP-1 derivative.

Although not wishing to be bound by any one theory, it is currently believed that GLP-1 promotes skin regeneration or hair growth through the affinity of GLP-1, GLP1(7-37) or GLP-1(7-36) amide for binding to the GLP-1 receptor (GLP-1R).

Hence, the present invention is also directed to a method of identifying an agent that regenerates skin cells comprising i) obtaining GLP-1R; ii) contacting a compound with the GLP-1R; and iii) determining whether the compound binds the GLP-1R; wherein the compound that binds GLP-1R is an agent that regenerates skin cells. Such binding experiments can be performed, for example, with¹²⁵I-GLP-1 binding to GLP-1R expressed on the surface of HEK 293 cells stably transfected with a GLP-1R expression plasmid.

The present invention is also directed to a method of identifying an agent that promotes hair growth comprising i) obtaining GLP-1R; ii) contacting a compound with the GLP-1R; and iii) determining whether the compound binds the GLP-1R; wherein the compound that binds GLP-1R is an agent that promotes hair growth. Such binding studies can be performed, for example, by preparing transgenic mice with expression of GLP-1R directed to skin by K10 or K14 promoters. The mice can be shaved and/or depilated. Hair regrowth can be measured in the presence and absence of topically applied GLP-1.

The present invention is also directed to treating diabetes. In particular, the present invention is directed to obtaining endothelial or skin cells; culturing the cells in dedifferentiating medium, and exposing the cultured cells to GLP-1, or a GLP-1 fragment or derivative thereby causing the cells to redifferentiate into insulin producing cells. Such cells can be administered to patients suffering from diabetes.

Epithelial cells from diverse tissue sources, including skin, can be dedifferentiated. Treatment of dedifferentiated cells with GLP-1 during redifferentiation leads to the expression and production of insulin in these cells. As a consequence, the use of GLP-1 or GLP-1 receptor agonists can be used in the redifferentiation of cells derived from diabetic subjects in order to make insulin-producing cells for autotransplantation or allotransplantation into patients with type 1 or type 2 diabetes mellitus.

### GLP-1, GLP-1 Derivatives or Fragments

GLP-1, GLP-1 derivatives and GLP-1 fragments are all useful according to the methods of the present invention. A GLP-1 derivative can have the same or different number of amino acid residues in its sequence as GLP-1, but will have at least one different or modified amino acid residue as compared to GLP-1. A GLP-1 fragment will have at least one less amino acid residue in its sequence as compared to GLP-1, but optionally can also have different or modified amino acid residues as compared to GLP-1.

Unless otherwise indicated, the terms “GLP-1,” and “GLP-1(1-37)” as used herein have the same meaning, are used interchangeably, and refer to human glucagon like peptide-1. The amino acid sequence for mammalian GLP-1 has been reported by several researchers (Lopez, L. C. et aL, Proc. Natl. Acad. Sci., U.S.A. 80:5485-5489 (1983); Bell, G. I. et al., Nature 302:716-718 (1983); Heinrich, G. et al., EndocrinoL 115:2176-2181 (1984)). The sequence of human GLP-1 is described in U.S. Pat. No. 6,849,708, which is hereby incorporated by reference in its entirety. In particular, the sequence of human GLP-1 (1-37) (SEQ ID NO: 1) is His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg Gly (wherein His is residue #1, and the terminal Gly is residue #37).

The present invention is directed to using GLP-1, GLP-1 fragments or GLP-1 derivatives for regenerating skin growth, promoting hair growth or for redifferentiating endothelial or skin cells into insulin generating or secreting cells. Such peptides include both synthetic and naturally occurring amino acid sequences derivable from a naturally occurring amino acid sequence. The GLP-1 can be human GLP-1, or a homologous sequence derived from human or another animal species. GLP-1 derivatives and GLP-1 fragments include homologous sequences derived from human or another animal species. Such GLP-1 derivatives and GLP-1 fragments include sequences which are about 40%, 45%, 50%, 55%, 60%, 65%, 70%, 75%, 80%, 85%, 90%, 91%, 92%, 93%, 94%, 95%, 96%, 97%, 98%, or 99% identical to human GLP-1(1-37), or a fragment thereof, such as human GLP-1(7-37) or human GLP-1(7-36). The degree of homology of the homologous sequences derived from human or another animal species can also fall between any two of the aforementioned percentages.

The percentage homology of a sequence homologous to GLP-1 is measured by comparison of the homologous sequence to GLP-1(1-37) or a fragment of GLP-1(1-37) having the same number of amino acid residues as the homologous sequence. If the homologous sequence is longer than GLP-1(1-37), then the percentage homology of the homologous sequence is measured by comparison of a 37 amino acid fragment of the homologous sequence to GLP-1(1-37).

GLP-1 fragments include both N- or C-terminal human GLP-1 fragments, or GLP-1 fragments from other animal species. As used herein, the notation of GLP-1(1-37) refers to a GLP-1 polypeptide having all amino acids from 1 (N-terminus) through 37 (C-terminus). Similarly, GLP-1(7-37) refers to a GLP-1 polypeptide having all amino acids from 7 (N-terminus) through 37 (C-terminus). Similarly, GLP-1(7-36), GLP-1(7-35) and GLP-1(7-34) refer to a GLP-1 polypeptide having all amino acids from number 7 (N-terminus) through number 36, 35 or 34 (C-terminus) respectively.

In one embodiment, GLP-1, GLP-1 fragments, or GLP-1 derivatives are synthesized by conventional means, such as by the well-known solid-phase peptide synthesis described by Merrifield, J. M. (Chem. Soc. 85:2149 (1962)), and Stewart and Young (Solid Phase Peptide Synthesis (Freeman, San Francisco, 1969), pages 27-66), which are incorporated by reference herein. However, it is also possible to obtain fragments of the proglucagon polypeptide, or of GLP-1, by fragmenting the naturally occurring amino acid sequence, using, for example, a proteolytic enzyme. Further, it is possible to obtain the desired fragments of the proglucagon peptide or of GLP-1 through the use of recombinant DNA technology, as disclosed by Maniatis, T., et al., Molecular Biology: A Laboratory Manual, Cold Spring Harbor, N.Y. (1982), which is hereby incorporated by reference.

The present approach includes peptides which are derivable from GLP-1(1-37). A peptide is said to be “derivable from a naturally occurring amino acid sequence” if it can be obtained by fragmenting a naturally occurring sequence, or if it can be synthesized based upon a knowledge of the sequence of the naturally occurring amino acid sequence or of the genetic material (DNA or RNA) which encodes this sequence.

Included within the scope of the present approach are those molecules which are said to be “derivatives” of human GLP-1(1-37), or GLP-1 from another animal species. Such a “derivative” shares substantial homology with GLP-1(1-37) or a similarly sized fragment of GLP-1(1-37).

Non limiting GLP-1 derivatives useful according to the methods of the present invention include the following (polypeptides represented using single letters to denominate the amino acid residues; source of sequence, where known, is stated prior to the sequences):

Galus galus:
(SEQ ID NO: 2)
HSEFERHAEGTYTSDITSYLEGQAAKEFIAWLVNGRG
or
(SEQ ID NO: 3)
HAEGTYTSDITSYLEGQAAKEFIAWLVNGRG
or
(SEQ ID NO: 4)
HSEGTFTSDFTRYLDKMKAKDFVHWLI;
Agkistrodon piscivorus:
(SEQ ID NO: 5)
HAEYERHADGTYTSDISSYLEGQAAKEFIAWLVNGRG
or
(SEQ ID NO: 6)
HADGTYTSDISSYLEGQAAKEFIAWLVNGRG;
Heloderma suspectuni (gila monster):
(SEQ ID NO: 7)
HAEYERHADGRYTSDISSYLEGQAAKEFIAWLVNGRG
or
(SEQ ID NO: 8)
HADGRYTSDISSYLEGQAAKEFIAWLVNGRG
or
(SEQ ID NO: 9)
HGEGTFTSDLSKQMEEEAVRLFIEWLKNG
or
(SEQ ID NO: 10)
SKIKRHGEGTFTSDLSKQMEEEAVRLFIEWLKNG;
Heloderma horridum (Mexican beaded lizard):
(SEQ ID NO: 11)
HSDGTFTSDLSKQMEEEAVRLFIEWLKNG
or
(SEQ ID NO: 12)
SKIKRESDGTFTSDLSKQMEEEAVRLFIEWLKNG;
Neoceratodus forsteri:
(SEQ ID NO: 13)
HSEIERHAEGTYTSDISSYLEGQAANEFVRWLLKGRG
or
(SEQ ID NO: 14)
HAEGTYTSDISSYLEGQAANEFVRWLLKGRG;
Protopterus dolloi:
(SEQ ID NO: 15)
HNEIERHAEGTYTSDISSYLEGQAVNEFIRWLMKGRG
or
(SEQ ID NO: 16)
HAEGTYTSDISSYLEGQAVNEFIRWLMKGRG;
Xenopus laevis:
(SEQ ID NO: 17)
EFERHAEGTYTNDVTEYLEEKAAKEFIEWLIKGK
or
(SEQ ID NO: 18)
YSRHAEGTFTNDMTNYLEEKAAKEFVGWLIKGR
or
(SEQ ID NO: 19)
DYERHAEGTFTSDVTQQLDEKAAKEFIDWLING
or
(SEQ ID NO: 20)
HAEGTFTNDMTNYLEEKAAKEFVGWLIKGR
or
(SEQ ID NO: 21)
HAEGTYTNDVTEYLEEKAAKEFIEWLIKGK
or
(SEQ ID NO: 22)
HAEGTFTSDVTQQLDEKAAKEFIDWLING
or
(SEQ ID NO: 23)
HSQGTFTSDYSKYLDSRRAQDFVQWLM
or
(SEQ ID NO: 24)
HADGSFTNDINKVLDIIAAQEFLDWVI;
Lophius americanus:
(SEQ ID NO: 25)
FRRHADGTYTSDVSSYLQDQAAKDFVSWLKAGRG;
Tetraodon nigroviridis:
(SEQ ID NO: 26)
FRRHADGTYTSDVSAYLQDQAAKEFVSWLKTGRG
or
(SEQ ID NO: 27)
HADGTYTSDVSAYLQDQAAKEFVSWLKTGRG
or
(SEQ ID NO: 28)
HSEGTFSNDYSKYLETRRAQDFVQWL
or
(SEQ ID NO: 29)
HADGTFTSDVSSYLKDQAIKDFVARLKAGQ;
Sebastes caurinus:
(SEQ ID NO: 30)
FRRHADGTYTSDVSSYLQDQAAKEFVYWLKTGRG
or
(SEQ ID NO: 31)
HADGTYTSDVSSYLQDQAAKEFVYWLKTGRG
or
(SEQ ID NO: 32)
HSEGTFSNDYSKYLETRRAQDFVQWL
or
(SEQ ID NO: 33)
HADGTFTSDVSSYLKDQAIKDFVNRLKSGQ;
Lophius americanus:
(SEQ ID NO: 34)
HADGTFTSDVSSYLKDQAIKDFVDRLKAGQ
or
(SEQ ID NO: 35)
HSEGTFSNDYSKYLEDRKAQEFVRWLMNNK;
Danio rerio:
(SEQ ID NO: 36)
RHADGTYTSDVSSYLQDQAAKEFVSWLKTGRG
or
(SEQ ID NO: 37)
HADGTYTSDVSSYLQDQAAKEFVSWLKTGRG
or
(SEQ ID NO: 38)
HAEGTYTSDVSSYLQDQAAQSFVAWLKSGQ
or
(SEQ ID NO: 39)
HSEGTFSNDYSKYLETRRAQDFVQWLMNAK
or
(SEQ ID NO: 40)
HAEGTYTSDVSSYLQDQAAQRFVARLKSGQ;
Hoplobatrachus rugulosus:
(SEQ ID NO: 41)
QFERHAEGTYTNDVTQFLEEKAAKEFIDWLIKGK
or
(SEQ ID NO: 42)
HAEGTFTSDMTSYLEEKAAKEFVDWLIKGR
or
(SEQ ID NO: 43)
HAEGTYTNDVTQFLEEKAAKEFIDWLIKGK;
Xenopus tropicalis:
(SEQ ID NO: 44)
YSRHAEGTFTNDMTNYLEEKAAKEFVGWLIKGR
or
(SEQ ID NO: 45)
DVERHAEGTYTNDVTEYLEEKAAKEFTRWLINGK
or
(SEQ ID NO: 46)
DYERHAEGTFTSDVTQHLDEKAAKEFIDWLING
or
(SEQ ID NO: 47)
HAEGTFTNDMTNYLEEKAAKEFVGWLIKGR
or
(SEQ ID NO: 48)
HAEGTYTNDVTEYLEEKAAKEFIEWLINGK
or
(SEQ ID NO: 49)
HAEGTFTSDVTQHLDEKAAKEFIDWLING
or
(SEQ ID NO: 50)
HSQGTFTSDYSKYLDSRRAQDFIQWLM;
Ictalurus punctatus:
(SEQ ID NO: 51)
RHADGTYTSDVSSYMQDQAAKFTVSWLKMGRG
or
(SEQ ID NO: 52)
HADGTYTSDVSSYMQDQAAKEFVSWLKMGRG
or
(SEQ ID NO: 53)
HSEGTFSNDYSKYLETRRAQDFIQWLMNSK;
Amphiuma tridactyhun:
(SEQ ID NO: 54)
HADGTLTSDISSFLEKQATKEFIAWLVSGRG;
bullfrog:
(SEQ ID NO: 55)
HADGTFTSDMSSYLEEKAAKEFVDWLIKGR;
Lophius americanus:
(SEQ ID NO: 56)
HADGTYTSDVSSYLQDQAAKDFVSWLKAGRG
or
(SEQ ID NO: 57)
HSEGTFSNDYSKYLETRRAQDFVQWL;
eel:
(SEQ ID NO: 58)
HAEGTYTSDVSSYLQDQAAKEFVSWLKTGR;
Bufo marinus:
(SEQ ID NO: 59)
HAEGTFTSDMTSFLEEKAAKEFVDWLIKGR
or
(SEQ ID NO: 60)
HAEGTYTNDVTQFLEEKAAKEFIDWLLKG
or
(SEQ ID NO: 61)
HADGSFTSDFNKALDIKAAQEFLDWII;
Oncorhynchus mylciss:
(SEQ ID NO: 62)
HADGTYTSDVSTYLQDQAAKDFVSWLKSGR;
Oncorhynchus tshawytscha:
(SEQ BD NO: 63)
HADGTYTSDVSTYLQDQAAKDFVSWLKSGR;
channel catfish:
(SEQ ID NO: 64)
HADGTYTSDVSSYLQDQAAKDFITWLKSGQ
or
(SEQ ID NO: 65)
HSEGTFSNDYSKYLETRRAQDFVQWLM;
Ictalurus pzinctatus:
(SEQ ID NO: 66)
HADGTYTSDVSSYLQDQAAKDFITWLKSGQ;
alligator gar:
(SEQ ID NO: 67)
HADGTYTSDVSSYLQDQAAKKFVTWLKQGQ
or
(SEQ ID NO: 68)
HSQGTFTNDYSKYLDTRRAQDFVQWLM;
Tilapia nilotica:
(SEQ ID NO: 69)
GTYTSDVSSYLQDQAAKEFVSWLKTGRG;
rainbow trout:
(SEQ ID NO: 70)
HADGTYTSDVSTYLQDQAAKDFVSWLKSG;
coho salmon:
(SEQ ID NO: 71)
HADGTYTSNVSTYLQDQAAKDFVSWLKSGR;
Scyliorhinus canicula:
(SEQ ID NO: 72)
HSEGTFTSDYSKYMDNRRAKDFVQWLM
smaller spotted catshark:
(SEQ ID NO: 73)
HSEGTFTSDYSKYMDNRRAKDFVQWLM
Polyodon spathula:
(SEQ ID NO: 74)
HADGTYTSDASSFLQEQAARDFISWLKKGQ
or
(SEQ ID NO: 75)
HSQGMFTNDYSKYLEEKSAKEFVEWLKNGK;
Squalus acanthias:
(SEQ ID NO: 76)
HTEGSYRDISSYLEAKAAKDFINWLIKGRG;
shorthorn sculpin:
(SEQ ID NO: 77)
HADGTFTSDVSSYLNDQAIKDFVAKLKSGK
or
(SEQ ID NO: 78)
HSEGTFSNDYSKYLETRRAQDFVQWL;
Petromyzon marinus:
(SEQ ID NO: 79)
HADGTFTNDMTSYLDAKAARDFVSWLAR
or
(SEQ ID NO: 80)
HSEGTFTSDYSKYLENKQAKDFVRWLMNAK
or
(SEQ ID NO: 81)
HSEGTFTSDYSKYLENKQAKDFVRWLM;
marbled electric ray:
(SEQ ID NO: 82)
HSEGTFTSDYSKYLDNRRAKDFVQWLM;
Hydrolagus colliei:
(SEQ ID NO: 83)
EEDYPNDFSSYLEAKAAKDFIDWLIKGQG;
Callorhynchus
(SEQ ID NO: 84)
HSEGTFSSDYSKYLDSRRAKDFVQWLM
or any of the following sequences:
(SEQ ID NO: 85)
HADGTYTSDVSSYLQDQAAKEFVSWLKTGRG;
(SEQ ID NO: 86)
HADGTYTSDVSSYLQDQAAKKFVTWLKQGQ;
(SEQ ID NO: 87)
HADGTYTSNVSTYLQDQAAKDFVSWLKSGR;
(SEQ ID NO: 88)
HADGTYTSDVSSYLQEQAAKDFITWLKSGQ;
(SEQ ID NO: 89)
HADGTFTSDVSSYLNDQAIKDFVAKLKSGK;
(SEQ ID NO: 90)
HAEGTYTSDISSFLRDQAAQNFVAWLKSGQ;
(SEQ ID NO: 91)
HADGTFTSDMSSYLEEKAAKEFVDWL1KGR;
(SEQ ID NO: 92)
HSEGTFSNDYSKYLETQRAQDFVQWLM;
(SEQ ID NO: 93)
HSEGTFSNDYSKYLETRRAQDFVQWLM;
(SEQ ID NO: 94)
HSQGTFTSDYSKYLDSKKAQEFVQWLM;
(SEQ ID NO: 95)
HSQGTFTSDYSKYLDNRRAQDFIQWLM;
and
(SEQ ID NO: 96)
HSEGTFSNDYSKYLETRRAQDFVQWL.

The derivatives of the present approach include GLP-1(1-37) fragments which, in addition to containing a sequence that is substantially homologous to that of a naturally occurring GLP-1(1-37) peptide can contain one or more additional amino acids at their amino and/or their carboxy termini. Thus, the present approach pertains to polypeptide fragments of GLP-1(1-37) that can contain one or more amino acids that are not present in a naturally occurring GLP-1(1-37) sequence.

Similarly, the present approach includes GLP-1(1-37) fragments which, although containing a sequence that is substantially homologous to that of a naturally occurring GLP-1(1-37) peptide can lack one or more additional amino acids at their amino and/or their carboxy termini that are naturally found on a GLP-1(1-37) peptide. Thus, the present approach pertains to polypeptide fragments of GLP-1(1-37) that lack one or more amino acids that are normally present in a naturally occurring GLP-1(1-37) sequence.

The present approach also encompasses the obvious or trivial variants of the above-described fragments which have inconsequential amino acid substitutions (and thus have amino acid sequences which differ from that of the natural sequence). Examples of obvious or trivial substitutions include the substitution of one basic residue for another (i.e. Arg for Lys), the substitution of one hydrophobic residue for another (i.e. Leu for Ile), or the substitution of one aromatic residue for another (i.e. Phe for Tyr), etc.

Other non-limiting examples of derivatives of GLP-1(1-37) that are useful in the methods of the present invention include GLP-1(7-37); GLP-1(7-36); GLP-1(7-35); GLP-1(7-34); and the des-G1y³⁷ amidated forms of these molecules. GLP-1(7-36) amide is another derivative or fragment of GLP-1(7-37) that is useful in the method of the present invention. Included as well are the use of additional amino acid residues added to such sequences in order to enhance coupling to carrier protein or amino acid residues added to enhance the ability of the GLP-1, GLP-1 derivative or GLP-1 fragment to stimulate hair growth or skin regeneration.

As is known in the art, the amino acid residues can be in their protected or unprotected form, using appropriate amino or carboxyl protecting groups. Useful cations are alkali or alkaline earth metallic cations (i.e., Na, K, Li, 1/2Ca, 1/2Ba, etc.) or amine cations (i.e., tetraalkylammonium, trialkylammonium, where alkyl can be C1-C12).

The variable length peptides can be in the form of the free amines (on the N-terminus), or acid-addition salts thereof. Common acid addition salts are hydrohalic acid salts, i.e., HBr, HI, or, more preferably, HCl.

GLP-1 derivatives also include the peptide sequence His-Ser-Asp-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-amide (SEQ ID NO: 97), as described by Eng et al., J Biol Chem. 265(33):20259-62 (1990); or the peptide sequence His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser amide (SEQ ID NO: 98), as described by Eng et al. J. Biol. Chem. 267(11): 7402-5 (1992).

GLP-1 derivatives also include GLP-1, GLP-1 derivatives or GLP-1 fragments modified so as to be covalently bound to or ionically associated with a carrier or targeting moiety. The carrier or targeting moiety can facilitate dermal or cell membrane permeation, or enhance bioavailability of the GLP-1, GLP-1 derivative or GLP-1 fragment. The carrier moiety can be a lipophilic compound or a surfactant -covalently bound to or ionically associated with the GLP-1, GLP-1 derivative or GLP-1 fragment. The targeting moiety can be any moiety recognized by a transmembrane or intracellular receptor protein.

Other non-limiting examples of GLP-1 derivatives useful in practicing the present invention include deletion sequences of GLP-1(1-37), the natural and non-natural amino acid residue substitutes thereof, the C-terminus carboxamides thereof (e.g., GLP-1(7-36)amide), the C-terminus esters thereof, the C-terminus ketones thereof, the N-terminus modifications thereof, or any mixture thereof.

Other non-limiting examples of GLP-1 derivatives useful in practicing the present invention include exendin-3, exendin-4, D-Ala⁸-GLP-1 (7-3 6)-Lys³⁷(ε-(AEEA)-MPA)-NH₂), (where AEEA refers to ([2-(2-amino)ethoxy)]ethoxy acetic acid) and MPA refers to maleimidopropionic acid).

Other non-limiting examples of GLP-1 derivatives useful in practicing the present invention include C-terminal salts, esters and amides of GLP-1 (1-37) where the salts and esters are defined as OM where M is a phaiivaceutically acceptable cation or a lower (C₁-C6) branched or unbranched alkyl group and the amides are defined as —NR₂ R₃ where R₂ and R₃ are the same or different and are selected from the group consisting of hydrogen and a lower (C1-C6) branched or unbranched alkyl group.

Other non-limiting examples of GLP-1 derivatives useful in practicing the present invention include an amino acid sequence of a formula: Rᵢ-X-Glu-Gly-Thr-Phe-Thr-Ser-Asp-Val-Ser-Ser-Tyr-Leu-Y-Gly-Gln-Ala-Ala-Lys-Z-Phe-Ile-Ala-Trp-Leu-Val-Lys-Gly-Arg-R₂ (SEQ ID NO: 99), wherein: R₁ is selected from the group consisting of Lhistidine, D-histidine, desamino-histidine, 2-amino-histidine, beta-hydroxy-histidine, homohistidine, alpha-fluoromethyl-histidine, and alpha-methylhistidine; X is selected from the group consisting of Gly, Val, Thr, He, and alpha-methyl-Ala; Y is selected from the group consisting of Glu, Gln, Ala, Thr, Ser, and Gly; Z is selected from the group consisting of Glu, Gln, Ala, Thr, Ser, and Gly; and R₂ is selected from the group consisting of NH₂, and Gly-OH.

Other non-limiting examples of GLP-1 derivatives useful in practicing the present invention include Gly⁸-GLP-1 (7-36)NH₂, Val⁸-GLP-1(7-37)OH, alpha-methyl-Ala⁸-GLP-1 (7-36)NH₂, Gly⁸-Gln²¹-GLP-1(7-37)OH, Val⁸-GLP-1(7-37)OH, Gly⁸-GLP-1(7-37)OH, or Val⁸-GLP-1(7-37)OH.

Other non-limiting examples of GLP-1 derivatives useful in practicing the present invention include Gln⁹-GLP-1(7-37), D-Gln⁹-GLP-1(7-37), acetyl-Lys⁹-GLP-1 (7-37), GLP-1(7-36)NH₂, Gly⁸-GLP-1(7-36)NH₂, D-Thr⁹-GLP-1(7-37), Asn⁹-GLP-1 (7-3 7), D-Asn⁹-GLP-1 (7-37), Ser²²-Arg²³-Arg²⁴-Gln²⁶-GLP-1(7-37), Thr¹⁶-Lys¹⁸-GLP-1 (7-37), Lys¹⁸-GLP-1 (7-37), Arg²³-GLP-1(7-37), Arg²⁴-GLP-1(7-37) and the like, and derivatives thereof including, for example, acid addition salts, carboxylate salts, lower alkyl esters, and amides.

Other non-limiting examples of GLP-1 derivatives useful in practicing the present invention include GLP-1(1-36)-Lys³⁷(ε-(AEEA)ₙ, -MPA)-NH₂; GLP-1(7-36)-Lys³⁷(ε(AEEA)ₙ MPA)-NH₂; exendin-4(1-39)-Lys⁴⁰(ε-(AERE)ₙ-MPA)-NH₂; exendin-3 (1-39)-Lys⁴⁰(ε-(AEEA)ₙ-MPA)-NH₂; exendin-4(1-39)-Lys⁴⁰ (ε-(AEEA)ₙ-MPA)-NH₂; Lys²⁶ ε(AEEA)ₙMPA-GLP-1 (7-36)-NH₂; GLP-1(7-36)-EDA-MPA; and exendin 4(1-39)-EDAMPA, wherein n=0-2.

Other non-limiting examples of GLP-1 derivatives useful in practicing the present invention include one or more of the following: GLP-1(1-36)-Lys³⁷(ε-MPA)-NH₂, GLP-1 (1-36)-Lys³⁷(ε-AEEA-AEEA-MPA)-NH₂, GLP-1 (7-36)-Lys³⁷(ε-MPA)-NH₂, GLP-1 (7-36)-Lys³⁷(ε-AEEA-AEEA-MPA)-NH₂, exendin-4(1-39)-Lys⁴⁰ (ε-MPA)-NH₂, exendin-4 (1-39)-Lys⁴⁰ (ε-AEEA-AEEA-MPA)-NH2, exendin-3(1-39)-Lys⁴⁰ (ε-MPA)-NH₂, and exendin-3(1-39)-Lys⁴⁰ (ε-AEEA-AEEA-MPA).

Other non-limiting examples of GLP-1 derivatives useful in practicing the present invention include a GLP-1 derivative comprising at least one modification selected from the group consisting of: a) substitution of a neutral amino acid, arginine, or a D form of lysine for lysine at position 26 and/or 34 and/or a neutral amino acid, lysine, or a D form of arginine for arginine at position 36; b) substitution of an oxidation-resistant amino acid for tryptophan at position 31; c) substitution according to at least one of: Y for V at position 16; K for S at position 18; D for E at position 21; S for G at position 22; R for Q at position 23; R for A at position 24; or Q for K at position 26; d) substitution comprising at least one of: (i) an alternative small neutral amino acid for A at position 8; (ii) an alternative acidic amino acid or neutral amino acid for E at position 9; (iii) an alternative neutral amino acid for G at position 10; or (iv) an alternative acidic amino acid for D at position 15; and e) substitution of an alternative neutral amino acid or the D or Nacylated or alkylated form of histidine for histidine at position 7.

Other non-limiting examples of GLP-1 derivatives useful in practicing the present invention include a GLP-1 analog comprising a substitution of glycine, serine, cysteine, threonine, asparagine, glutamine, tyrosine, alanine, valine, isoleucine, leucine, methionine, phenylalanine, arginine, or D-lysine for lysine at position 34.

Other non-limiting examples of GLP-1 derivatives useful in practicing the present invention include a GLP-1 derivative wherein (a) the 6-amino group of Lys at position 26 is substituted with a lipophilic substituent, optionally via a spacer, (b) the lipophilic substituent is (i) CH₃ (CH₂)ₙCO— wherein n is 6, 8, 10, 12, 14, 16, 18, 20 or 22, (ii) OOC(CH₂)ₘC— wherein m is 10, 12, 14, 16, 18, 20 or 22, or (iii) lithochoyl, and (c) the spacer is (i) an unbranched alkane a, w-dicarboxylic acid group having from 1 to 7 methylene groups, (ii) an amino acid residue except Cys, or (iii) γ-aminobutanoyl.

Other non-limiting examples of GLP-1 derivatives useful in practicing the present invention include a GLP-1 derivative wherein (a) the ε-amino group of Lys at position 26 is substituted with a lipophilic substituent optionally via a spacer, and (b) the lipophilic substituent is (i) CH₃ (CH₂)ₙCO— wherein n is 6, 8, 10, 12, 14, 16, 18, 20 or 22 (ii) OOC(CH₂)ₘCO— wherein m is 10, 12, 14, 16, 18, 20 or 22, or (iii) lithocholyl, and (c) the spacer is an amino acid residue except Cys, or the spacer is γ-aminobutanoyl.

Other non-limiting examples of GLP-1 derivatives useful in practicing the present invention include one of the following: GLP-1(1-36)-Lys³⁷ (ε-MPA)-NH₂; GLP-1 (1-36)-Lys³⁷ (ε-AAEA-AEEA-MPA)-NH₂; GLP-1 (7-36)-Lys³⁷ (ε-MPA)-NH₂; GLP-1 (7-36)-Lys³⁷-(ε-AEEA-AEEA-MPA)-NH₂; D-Ala²GLP-1 (7-36)-Lys³⁷ (ε-MPA)-NH₂; exendin-4 (1-39)-Lys⁴⁰ (ε-MPA)-NH₂; exendin-4 (1-39)-Lys⁴⁰ (ε-AEEA-AEEA-MPA)-NH₂; exendin-3 (1-39)-Lys⁴⁰ (ε-MPA)-NH₂; exendin-3 (1-39)-Lys⁴⁰ (ε-AEEA-AEEA-MPA)-NH₂; Lys² (ε-MPA)GLP-1(7-36)-NH₂; GLP-1 (7-36)-EDA-MPA and exendin-4 (1-39)-EDA-MPA.

Other GLP-1 derivatives or fragments useful according to the methods of the present invention are described in U.S. Pat. Nos. 6,911,324; 6,852,690; 6,849,708; 6,828,303; 6,821,949; 6,720,407; 6,703,365; 6,703,359; 6,593,295; 6,583,111; 6,528,486; 6,514,500; 6,458,924; 6,329,336; 6,309,633; 6,268,343; and 5,545,618; all of which are hereby incorporated by reference.

### Administration to Mammals

The present invention finds application in all mammalian species, including both humans and animals. In humans, the compounds used in the present invention can be applied for example, to any desired area including the head, pubic area, upper lip, eyebrows, and eyelids.

In animals raised for their hair or pelts, e.g. sheep or mink, the compounds can be applied over the entire surface of the body to improve the hair growth. The compounds can also be applied to domestic pets (e.g., cats and dogs) or to farm animals (e.g., cows, horses, pigs) to improve their hair growth. The process can also be used for cosmetic reasons in animals, e.g. applied to the skin of dogs and cats having bald patches due to mange or other diseases. In a preferred embodiment, the compounds of the present invention are applied to animals in a season when animals typically grow denser hair, such as fall or winter, to augment their seasonally thicker coats. However, the compounds of the present invention can also be applied to animals in other seasons as well.

### Modes of Administration

GLP-1, its derivatives or fragments can be administered topically by incorporation into a suitable pharmaceutical carrier, and applied at the site for exertion of local action. Accordingly, such topical compositions include those pharmaceutical forms in which the compound is applied externally by direct contact with the skin surface to be treated. Conventional phaimaceutical forms for this purpose include ointments, lotions, creams, pastes, jellies, sprays, aerosols, and the like. The term “ointment” embraces formulations (including creams) having oleaginous, absorption, water-soluble and emulsion-type bases, e.g., petrolatum, lanolin, polyethylene glycols, as well as mixtures of these. The percentage by weight of the GLP-1, its derivative or fragment utilized can range from about 0.01% to about 20.0% of the pharmaceutical preparation.

The GLP-1, GLP-1 fragment or GLP-1 derivative can also be mixed with a cosmetic, suntan lotion, moisturizing or hydrating lotion or other body lotion.

The present invention is also directed to applying GLP-1, its derivatives or fragments by laser or pressure waves as described in U.S. Pat. Nos. 4,775,361; 5,614,502; 5,658,892; 6,251,099; 6,562,004; and 6,689,094, which are herein wholly incorporated by reference. GLP-1, its derivatives or fragments can also be administered using micro needles such as those described in U.S. Pat. Nos. 6,881,203; 6,808,506; 5,879,326; 6,494,865; 5,997,501; 4,886,499; 6,183,434; 5,250,023; 5,527,288; and 3,595,231, which are herein wholly incorporated by reference. GLP-1, its derivatives or fragments can also be administered as a component of artificial skin such as those describe in U.S. Pat. Nos. 6,110,208; 6,733,530; 5,326,356; 4,912,049; and 5,650,164. GLP-1, its derivatives or fragments can also be administered via intradermal injection.

### Formulations and Compositions

As used herein, “GLP-1 containing composition” or “GLP-1 containing formulation” refers to compositions or formulations containing one or more GLP-1, GLP-1 fragment or GLP-1 derivative. Therapeutic compositions of the present approach can consist solely of GLP-1, GLP-1 fragments or GLP-1 derivatives. Preferably, the compositions will be combined in admixture with a pharmaceutically acceptable carrier vehicle.

Suitable vehicles and their formulation, inclusive of other human proteins, e.g., human serum albumin, are described for example in Remington's Pharmaceutical Sciences (16th Ed., A. Oslo Ed. Mack, Easton, Pa. (1980)). In order to form a pharmaceutically acceptable composition that is suitable for effective administration to a patient in need of such composition, such compositions will contain an effective amount of GLP-1, one or more GLP-1 fragments or one or more GLP-1 derivatives, together with a suitable amount of carrier vehicle.

The GLP-1, GLP-1 fragments or GLP-1 derivatives of such compounds will preferably have been purified so as to be substantially free of natural contaminants. A material is said to be “substantially free of natural contaminants” if it has been substantially purified from materials with which it is normally and naturally found. Examples of natural contaminants with which GLP-1, GLP-1 fragments or GLP-1 derivatives might be associated are: other peptides, carbohydrates, glycosylated peptides, lipids, membranes, etc. A material is also said to be substantially free of natural contaminants if these contaminants are substantially absent from a sample of the material.

Compositions containing GLP-1, GLP-1 fragments or GLP-1 derivatives can be administered topically, intradermally or sub-cutaneously at dosages in the range of from about 1 pg/kg to 1,000 μg/kg body weight, although a lower or higher dosage can be administered. The dosages can also be in the following ranges: 1 pg/kg to 1 μg/kg body weight, 1 pg/kg to 0.1 μg/kg body weight, 10 μg/kg to 1000 μg/kg body weight, 100 μg/kg to 1000 μg/kg body weight or from 100 μg/kg to 500 μg/kg body weight. The required dosage will depend upon the severity of the condition of the patient, and upon such criteria as the patient's height, weight, sex, age, and medical history. The dose will also depend upon whether the compound of the present approach is being administered in a veterinary setting to an animal or to a human patient.

For the purpose of parenteral administration, compositions containing GLP-1,GLP-1 fragments or GLP-1 derivatives can be dissolved in distilled water and the pH-value can be adjusted to about 6 to 8. In order to facilitate the lyophilization process resulting in a suitable product, lactose can be added to the solution. Preferably, the solution is then filtered sterilized, introduced into vials, and lyophilized. The concentration of the GLP-1, GLP-1 fragments or GLP-1 derivatives in these compositions can vary from 10⁻¹²M to 10⁻⁵M.

Additional pharmaceutical methods can be employed to control the duration of action. Controlled release preparations can be achieved by the use of polymers to complex or adsorb the GLP-1, GLP-1 fragments or GLP-1 derivatives. The controlled delivery can be exercised by selecting appropriate macromolecules (for example, polyesters, polyamino acids, polyvinyl pyrrolidone, ethylenevinylacetate, methylcellulose, carboxymethylcellulose, and protamine sulfate) and the concentration of macromolecules as well as the methods of incorporation in order to control release. Another possible method to control the duration of action by controlled release preparations is to incorporate the GLP-1, GLP-1 fragments or GLP-1 derivatives into particles of a polymeric material such as polyesters, polyamino acids, hydrogels, poly (lactic acid) or ethylene vinylacetate copolymers. Alternatively, instead of incorporating the GLP-1, GLP-1 fragments or GLP-1 derivatives into these polymeric particles, it is possible to entrap these derivatives in microcapsules prepared, for example, by coacervation techniques or by interfacial polymerization, for example, hydroxymethylcellulose or gelatin-microcapsules and poly (methylmethacrylate) microcapsules, respectively, or in colloidal drug delivery systems, for ‘example, liposomes, albumin microspheres, microemulsions, nanoparticles, and nanocapsules or in macroemulsions. Such teachings are disclosed in Remington's Pharmaceutical Sciences (1980). The GLP-1, GLP-1 fragments or GLP-1 derivatives can be formulated for delivery from an adhesive patch.

It is possible to enhance the biological half-life of the GLP-1, GLP-1 fragments or GLP-1 derivatives, and, thus, to increase the retention or stability of the derivatives in a recipient, by bonding such derivatives to one or more chemical “moieties” to thereby produce a compound which can be recognized and processed within a recipient to yield a GLP-1, GLP-1 fragment or GLP-1 derivative. The “moieties” of such compounds can include one or more lipids, carbohydrates, amino acid residues, etc. A preferred “moiety” is an amino acid residue. The most preferred “moiety” is a peptide. The moiety can be bound to the amino or carboxy terminal residue of the GLP-1, GLP-1 fragment or GLP-1 derivative.

### Combination Therapy

The present invention also relates to improved methods and compositions for promoting hair growth by the concomitant administration of GLP-1, GLP-1 fragments or GLP-1 derivatives with potassium channel openers such as those described by U.S. Pat. No. 5,578,599, which is hereby incorporated by reference in its entirety. Non-limiting examples of agents with which GLP-1, GLP-1 fragments or GLP-1 derivatives can be coadministered include minoxidil (Upjohn Company, Kalamazoo, Mich.) cromakalim, pinacidil, or a compound selected from the classes of potassium channel openers such as s-triazine derivatives, benzopyran derivatives, pyridinopyran derivatives and thiane-1-oxide compounds; and a 5a-reductase inhibitors, 1713-(N-tert-butylcarbamoyl)-4-aza-5-aandrost-1-en-3-one.

Minoxidil (6-amino-1,2-dihydro-l-hydroxy-2-imino-4-piper-idinopyrimidine), or its analogs that can be combined with GLP-1, GLP-1 fragments or GLP-1 derivatives according to the present approach are described in U.S. Pat. Nos. 3,382,247, 3,451,451, 3,461,461, 3,644,354, 4,139,619 or 4,596,812; and J. M. McCall, et al., J. Org. Chem., 40: 3304 (1975), which are herein incorporated by reference in their entirety. Related sulfoxypyrimidinium, -pyridinium, and -triazinium compounds that can be combined with GLP-1, GLP-1 fragments or GLP-1 derivatives according to the present approach are described in U.S. Pat. No. 4,287,338, which are herein incorporated by reference.

GLP-1, GLP-1 fragments or GLP-1 derivatives can also be used according to the present invention in combination with one or more hormones believed to be involved in the regulation of folliculogenesis. Several hormones and hormone receptors are known to be involved in the regulation of folliculogenesis (McElwee, K. and Hoffmann, R. Eur J Dermatol 10:341-50 (2000); Alonso, L. C. and Rosenfield, R. L. Horm. Res. 60:1-13 (2003)). In addition to the non-peptidic hormones and their nuclear receptors—androgens, estrogens, retinoids, and thyroid hormone—several peptide hormones have been implicated in hair growth. These peptide hormones and their cognate receptors include the pituitary hormones prolactin and somatotropin, insulin and insulin growth factor-1 (Su, H. Y. et al., Dermatol. Online J. 5:1 (1999)), the angiotensin neuropeptides (Takeda, H. et al, Br. J. Dermatol. 147:276-80 (2002)), corticotropin-releasing hormone and proopiomelanocortin derived peptides (Pans, R. et al., Ann. NY. Acad. Sci. 885:350-63 (1999); Kauser, S. et al, J. Invest. Dermatol. 123:184-95 (2004); Kono, M. et al., Faseb. 1 15:2297-9 (2001); Mazurkiewicz, J. E. et al., J. Histochem. Cytochem. 48:905-14 (2000)), substance P (Arck, P. C. et al., Am. J. Pathol. 162:803-14 (2003); Peters, E. M., J. Invest. Dermatol. 116:236-45 (2001)), calcitonin gene related peptide (Peters, E. M. et al., J. Invest. Dertnatot 116:236-45 (2001); Hagner, S. et al., Peptides 23:109-16 (2002)), and members of the glucagon superfamily of peptide hormones, pituitary adenylyl cyclase activating peptide and vasoactive intestinal peptide (Fischer, T. C. et al., J. Cutan. Pathol. 29:65-71 (2002); Cheng-Chew, S.B. and Leung, P. Y., Histochem. Cell Biol. 105:443-52 (1996)).

### EXAMPLES

The examples described herein demonstrate that GLP-1 and its receptor are important regulators of skin development and folliculogenesis.

### Reagents

Unless otherwise stated below, the reagents used in the Examples were obtained as follows:

Tissue culture medium RPMI 1640, penicillin/streptomycin, and B-27 supplement were obtained from Invitrogen Corporation (Carlsbad, Calif.). bFGF and EGF were obtained from Sigma (St. Louis, Mo.). Protease inhibitor cocktail tablets were obtained from Boehringer Mannheim Corporation (Mannheim, Germany). The RNeasy Mini Kit for RNA extraction and purification was obtained from Qiagen Inc.—U.S.A. (Valencia, Calif.). Oligo dT and Superscript II reverse transcriptase were obtained from Invitrogen Corp. (Carlsbad, Calif.). Taq DNA polymerase was obtained from Takara Biomedical, Inc. (Berkley, Calif.). Phospho-P42 MAPK mouse monoclonal antibody and P44/42 MAPK rabbit polyclonal antibody were obtained from Cell Signaling Technology, Inc. (Beverly, Mass.). Mouse anti-rat nestin monoclonal antibody was obtained from BD Biosciences Pharmingen (San Diego, Calif.). Guinea pig anti-human insulin and goat anti-rat C-peptide sera were obtained from Linco Research, Inc. (St. Charles, Mo.). Rabbit anti-GLP-1 receptor and anti-GLP-1 antibody were generated in-house (Heller, R. S. et al., Diabetes 46:785-91 (1997)). Cy2 and Cy3 secondary antibodies were obtained from Jackson Laboratories (Bar Harbor, Me.). The Mouse-onMouse kit was obtained from Vector Laboratories (Burlingame, Calif.). Fluorescent mounting medium was obtained from Kirkegaard & Perry Laboratories (Gaithersburg, Md.).

### Animals

Unless otherwise stated below, the animals used in the Examples were obtained as follows:

C57 BL/6 mice were obtained from Jackson Laboratories and were bred and maintained in the Massachusetts General Hospital (MGH) animal care facility. They were used for obtaining skin samples and for deriving cultured stem/progenitor cells for immunocytochemistry, reverse transcriptase-polymerase chain reaction (RT-PCR), and Western immunoblotting. Animal experiments were conducted in accordance with National Institutes of Health (NIH) guidelines for the care and use of laboratory animals and with approval from the Massachusetts General Hospital Institutional Animal Care and Use Committee.

### Tissue Culture

Unless otherwise stated below, tissue culture was prepared for use in the Examples as follows:

Newborn C57 BL/6 mice were euthanized with carbon dioxide. Skin tissues were cleansed with 70% ethanol and removed from the abdomen and back by careful dissection. After rinsing twice with phosphate-buffered saline (PBS), the tissues were minced into small pieces, washed three times with PBS, digested with 0.25% trypsin at 37° C. for 40 min, and incubated with 0.1% DNase for 1 minute at room temperature (RT). After washing twice with PBS and once with RPMI 1640 containing penicillin/streptomycin/fungizone, dissociated cells were resuspended in cell expansion medium, (RPMI 1640 containing 10% fetal bovine serum (FBS), 20 ng/ml epidermal growth factor (EGF), 4 Ong/ml basic fibroblast growth factor (bFGF) and poured through a cell strainer. The cells were initially cultured undisturbed at 37° C. for 10 days. Adherent cell cultures were maintained in continuous passage by trypsinization and replating every 3-5 days.

For in-vitro differentiation, after passaging for one month, mouse skin-derived cells were freshly plated and overlaid with differentiation medium composed of RPMI1640 containing 1% HEPES, 1% penicillin/streptomycin/fungizone, 1% pyruvate, 2% B-27 supplement, 2% FBS, and 50nM GLP-1. The cultures were incubated at 37° C. for two weeks prior to analysis by immunocytochemistry. MINE cells, provided by J. Miyazaki, were cultured as described (Miyazaki, J. et al., Endocrinology 127:126-32 (1990)).

### RT-PCR

Unless otherwise stated below, reverse transcriptase-polymerase chain reaction (RT-PCR) was conducted in the Examples as follows:

Total cellular and tissue RNA was extracted using the RNeasy Mini Kit. cDNA synthesis was performed using oligo dT and Superscript II reverse transcriptase following the manufacturer's protocol. PCR was carried out using 2 gl of cDNA template, 1× buffer (Takara), 0.2 uM dNTP, 300 nM concentration of each oligonucleotide primer, and 2.5 U thermostable Taq polymerase in a 50 ul reaction volume. The GLP1-R primers used were: 1 foward (1f) atggccagcaccccaagcctcctgcgcct (SEQ ID NO: 100) and 1 reverse (1r) actcttcacactccgacaggtccctccag (SEQ ID NO: 101), 2f ggtaccacggtgtccctctcagagacggt (SEQ ID NO: 102) and 2r gggcagccgtgctatacatccacttgagg (SEQ ID NO: 103), 3f cctgaggaacagctcctgtccctgtacat (SEQ ID NO: 104) and 3r gtgaacagettgatgaagcgtagggttcc (SEQ ID NO: 105), 4f tcggtgttctcggagcagcgcatcttcaa (SEQ ID NO: 106) and 4r ctcattgttgacaaagcagtacaggatag (SEQ ID NO: 107). The primers for proglucagon were: 1f agaagaagtcgccattgctgag (SEQ ID NO: 108) and 1r gttctctttccaggtttaccactacg (SEQ ID NO: 109), 2f agaagcgcatgaggaccctgatg (SEQ ID NO: 110) and 2r acgagatgttgtgaagatggttgtg (SEQ ID NO: 111). The nestin primers were: forward agccgaggaccagatacttga (SEQ ID NO: 112) and reverse gactectgactttccattctacc (SEQ ID NO: 113).

PCR amplifications were carried out for 40 cycles, consisting of 30 second denaturation at 94° C., 1 minute annealing at 57° C., and 1 minute extension at 72° C. The identifications of the amplified products seen on agarose gels were confirmed by cloning and sequencing.

### Immunohistochemistry and Immunocytochemistry

Unless otherwise stated below, immunohistochemistry and immunocytochemistry was conducted in the Examples as follows:

For immunochemistry, 7 μm frozen sections were prepared from newborn C57BL/6 mouse skin, and skin-derived tissue culture cells were grown on coverslips. Tissues/coverslips were fixed with 4% paraformaldehyde at RT for 15 min They were then rinsed with PBS containing 0.2% Triton X-100 and blocked with 3% normal donkey serum for 1 hour at RT. Tissues/coverslips were incubated with primary antibody at a 1:500 dilution at RT for 1 hour, rinsed with PBS containing 0.2% Triton X-100, and incubated with secondary (donkey) Cy2- or Cy3-conjugated antisera at RT for 1 hour. They were then washed with PBS and mounted with fluorescent mounting medium. Preadsorption of primary antibody with cognate peptide was carried out by adding a fivefold excess (by weight) of cognate peptide and incubating at room temperature for 1 hour. Flourescent images were obtained using a Carl Zeiss (New York, N.Y.) microscope equipped with a Spot digital camera from Diagnostic Instruments (Sterling Heights, Md.

In performing immunohistochemistry on mouse tissues, when using a mouse monoclonal primary antibody, the above protocol was altered to include use of the Mouse-on-Mouse kit, following the manufacturer's instructions.

### Western Blotting

Unless otherwise stated below, Western blots were conducted in the Examples as follows:

Cultures of C57B6/L mouse skin-derived cells were grown in 100 mm dishes to 80% confluence. Cultures were washed once with PBS and incubated with KRB buffer (115 mM NaCl, 5 mM KCl, 10 mM NaHCO3, 2.5 mM MgCl2, 2.5 mM CaCl2, 20 mM HEPES, pH 7.4) containing 0 5% bovine serum albumin (BSA) and 1 mM glucose at 37° C. for 1 hour. Cultures were then incubated with 1 OnM GLP1 in the setting of no (0 mM), low (2.8 mM) or high (16.7 mM) glucose. Proteins were extracted with lysis buffer (50 mM Hepes pH 7.6, 160 mM NaCl, 20 mM sodium pyrophosphate, 20 mM glycerophosphate, 10 mM NaF, 2 mM sodium vanadate, 10% Triton X-100, and protease inhibitor cocktail). Ten (10) i.tg of protein per lane was electrophoressed in 4-12% BisTris gels (Invitrogen Corp.) and transferred onto nitrocellulose membranes (Nitrobind, Osmotics, Inc., Minnetonka, Minn.) at 4° C. overnight. Membranes were blocked with 3% BSA in PBS with 0.2% Tween-80 for 1 hour at RT, incubated with mouse monoclonal anti-phospho-P42 MAPK antibody (1:2000 dilution) at 4° C. overnight, washed, and incubated with horseradish peroxidase—conjugated secondary antibody at RT for 1 hour. Immunostained proteins were detected using a the ECL chemiluminescence detection kit (Amersham Biosciences, Ltd., Buckinghamshire, England). Membranes were subsequently stripped with buffer (0.7% 13-mercaptoethanol, 2% SDS, 0.7% Tris HCl, pH 6.7) at 55° C. for 10 min, re-blocked with 3% dry milk in PBS for 1 hour at RT, and incubated with rabbit polyclonal anti-P42/44 MAPK antibody (1:1500 dilution) at 4° C. overnight Immunostaining and detection were then repeated as above.

### Example 1

### GLP-1 Stimulates Hair Growth

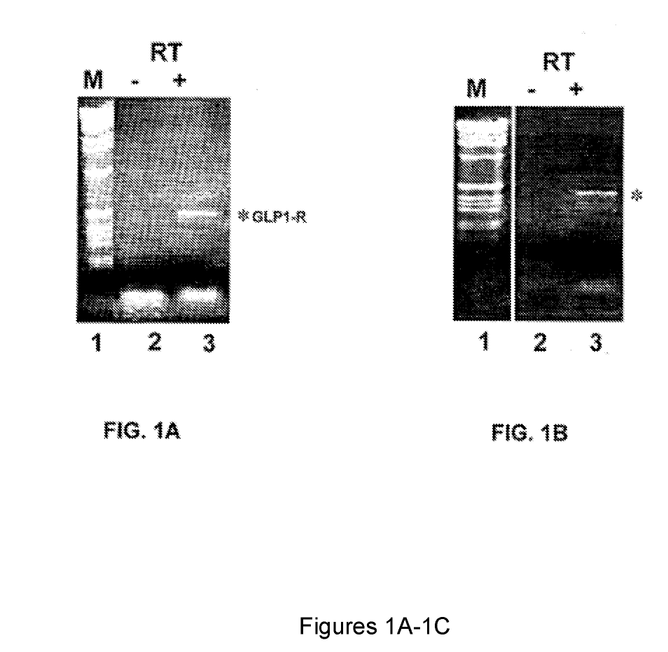

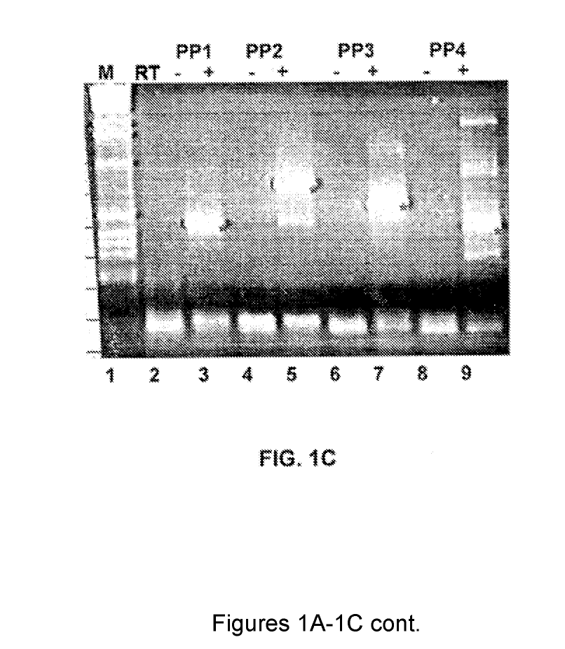

0.1mg (100 μg) of the following peptides were injected intradermally into the backs of the shaved New Zealand white rabbits: synthetic mammalian GLP-1 (1-37), synthetic GLP-1 peptide fragment containing amino acid sequence 16-30 of GLP-1 (1-37) (amino acid numbering starts from histidine 1 of mammalian GLP-1 (1-37), synthetic rat GLP-2 peptide fragment containing amino acid sequence 9-24 of rat GLP-2 (amino acid numbering starts at histidine 1 of the rat GLP-2 sequence), rat IP-2 and synthetic mammalian glucagon. Sequences for rat GLP-2, rat IP-2 and mammalian glucagon are well known in the art, and are provided, for example, in U.S. Pat. No. 6,849,708, which is hereby wholly incorporated by reference (see, e.g., FIG. 1 of U.S. Pat. No. 6,849,708). The total amount of peptide was administered in 10-20 intradennal injections as an emulsification in Freund's adjuvant. Each peptide was tested in four rabbits, except for GLP-1 (1-37) that was injected into 5 rabbits. This amount of peptide was administered at 3 time intervals (100 μg at each time interval), one week apart. Approximately 3-4 weeks after the third and last injection luxurious hair growth was observed only at the sites of injection in the rabbits (4 out of 5 rabbits) that were immunized with GLP-1 (1-37). This effect was not observed in any of the rabbits that were immunized with any other of the above described peptides. Repeat experiments at different seasons of the year gave variable results. The inventors observed enhanced hair growth upon intradermal injection of only GLP-1 (1-37) into rabbits.

### Example 2

### Identification of GLP-1R Expression in Mouse Skin and in Cultured Skin-Derived Cells

To investigate the expression of the GLP-1R in skin and in cultured skin-derived cells three experimental approaches were used: 1) examination of the expression of GLP-1R mRNA by RT-PCR, 2) Western immunoblot analysis of proteins in skin and cell extracts, and 3) in situ immunocytochemistry on skin tissue sections.

### Example 2A

GLP-1R mRNA was readily detected both in extracts of mouse skin and in cultures of skin-derived cells (FIGS. 1A-1C).

FIG. 1A depicts the expression of GLP-1 receptor (GLP-1R) mRNA in mouse skin detected by RT-PCR. Lane 1: molecular weight markers (M). Lane 2: without reverse transcriptase (RT-control). Lane 3: The asterisk shows the RT-PCR product of expected size when the primer pair was use with reverse transcriptase (RT+). Product of the anticipated size from PCR using primer pair 4f and 4r was cloned and sequenced to confirm identity.

FIG. 1B depicts the expression of GLP-1 receptor (GLP-1R) mRNA in cultured mouse skin-derived cells detected by RT-PCR. Lane 1: molecular weight markers (M). Lane 2: without reverse transcriptase (RT-control). Lane 3: The asterisk shows the RT-PCR product of expected size when the primer pair was used with RT (RT+). Product of the anticipated size from PCR using primer pair 4f and 4r was cloned and sequenced to confirm identity.

FIG. 1C depicts the expression of GLP-1 receptor (GLP-1R) mRNA in cultured mouse skin-derived cells using four different PCR primer pairs. Primer pair 4f and 4r (PP1=primer pair one) was used for lane 3 (lane 2 is a negative control wherein the primer pair of lane 3 is used without reverse transcriptase (RT−)); primer pair 3f and 3r (PP2=primer pair two) was used for lane 5 (lane 4 is a negative control wherein the primer pair of lane 5 is used without reverse transcriptase (RT−)); primer pair 2f and 2r (PP3=primer pair three) was used for lane 7 (lane 6 is a negative control wherein the primer pair of lane 7 is used without reverse transcriptase (RT−)); primer pair if and lr (PP4=primer pair four) was used for lane 9 (lane 8 is a negative control wherein the primer pair of lane 9 is used without reverse transcriptase (RT−)). The astersisk in lanes 3, 5, 7 and 9 show the RT-PCR product of expected size when the primer pairs are used with reverse transcriptase (RT+).

RNA was prepared from homogenized skin of 3 week-old C57B1/6 mice. Cultured skin-derived cells were prepared from 3 day old mice and RNA was prepared after culture for 30 days. Reverse transcription was carried out using oligo dT in the generation of the first strand of cDNA; PCR was carried out using mouse GLP-1 receptor-specific primers. 1% agarose gel electrophoresis of products.

The RT-PCR products generated were isolated from agarose gels, cloned, and sequenced to confirm their identity as that of the mouse GLP-1R (NCBI No. gi913834/gb575952.1).

### Example 2B

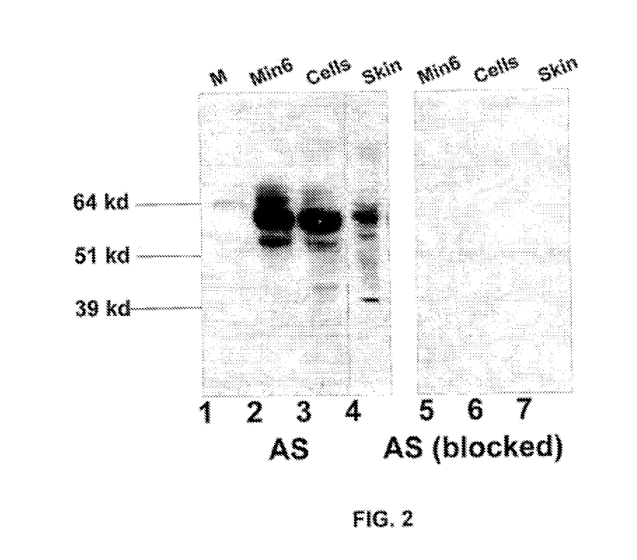

Western immunoblot analyses detected the GLP-1R protein of 56 kd in extracts of both skin and skin-derived cultured cells (FIG. 2, major band shown between 64 and 51 kd marks). FIG. 2 depicts the Western immunoblot of GLP-1 receptor from lysates of skin and primary skin cultures from C57B1/6 mice. Lanes 1-4: blot probed with rabbit polyclonal antiserum (AS) raised to a peptide of the GLP-1 receptor. Lanes 5-7: blot prepared in parallel and probed with the rabbit polyclonal antiserum preabsorbed with its cognate peptide (AS, blocked). Lane 1: molecular weight markers. Lanes 2,5: Min6 cell culture lysate (positive control). Lanes 3,6: C57b1/6 primary skin cell culture lysate. Lanes 4,7: C57b1/6 homogenized skin lysate. Anticipated molecular weight of murine GLP-1 receptor is 56 kd. SDS polyacrylamide gel electrophoresis of cell and tissue lysates was followed by blotting on nitrocellulose membranes and probing with rabbit polyclonal antibody specific for the GLP-1 receptor.

### Example 2C

Immunocytochemical staining of skin sections shows expression of the GLP-1R in localized regions of the hair follicles and the epidermis Immunostaining was performed using antiserum to αGLP-1R and normal rabbit serum or antiserum preabsorbed with its cognate peptide (αGLP-1R , blocked) (data not shown).

Staining is seen in cells of the mid to upper hair follicles as well as some cells of the basal layer of the epidermis. Only faint background staining is seen in the blocked antiserum and normal rabbit serum controls.

Frozen sections of skin from C57B1/6 mice were fixed with 4% paraformaldehyde and immunostained using rabbit polyclonal serum specific for the GLP-1 receptor. Binding was detected with a Cy-3-conjugated secondary antibody.

### Example 3

Detection by RT-PCR of the Expression of Proglucagon mRNA in Mouse Skin and in Cultured Skin-Derived Cells

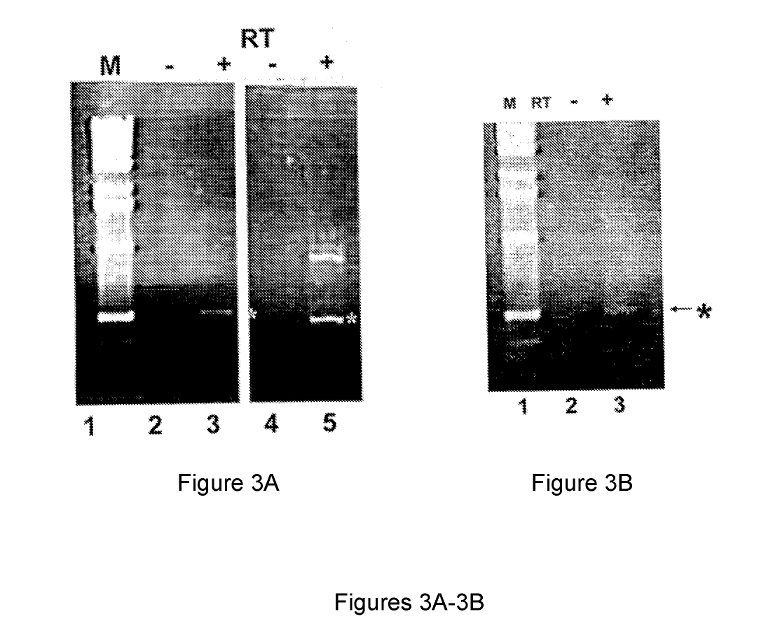

RT-PCR was performed on RNA extracted from the skin of 3 week old mice and from skin-derived cells prepared from 3 day old mice and maintained in continuous culture for 30 days (FIGS. 3A-3B).

FIG. 3A depicts RT-PCR of proglucagon mRNA in mouse skin. Lane 1: molecular weight marker (1kb ladder, Gibco/BRL). Lane 2: Control without reverse transcriptase (RT−) for primer pair one. Lane 3: RT-PCR product for primer pair one (expected product size marked with asterisk). Lane 4: Control without reverse transcriptase (RT−) for primer pair two. Lane 5: RT-PCR product for primer pair two (expected product size marked with asterisk).

FIG. 3B depicts RT-PCR of proglucagon mRNA in cultured skin-derived mouse cells. Lane 1: Molecular weight marker. Lane 2: Control without reverse transcriptase (RT−) with primer pair one. Lane 3: RT-PCR product for primer pair one (expected product size marked with asterisk).

The products generated by RT-PCR were cloned and sequenced, showing identity to the sequence of mouse proglucagon (NCBI No. gi15278023/gbB C012975.1).

RNA was prepared from skin and cultured cells as described in Example 2A. Reverse transcription was carried out using oligo dT in the generation of the first strand of cDNA; PCR was carried out using mouse glucagon-specific primers. 1% agarose gel electrophoresis of products.

Examples 2 and 3 indicate that proglucagon and the GLP-1 receptor (GLP-1R) are expressed in mouse skin cells. Both are also expressed in cultures of cells derived from mouse skin. Furthermore, these examples demonstrate that the expression of the GLP-1R in mouse skin is concentrated in hair follicles. This area is anatomically and topographically consistent with the bulge region of the hair follicle known to harbor stem cells. Hence, the expression of the GLP-1R in localized regions of the hair follicle indicates that these regions represent the bulge area of the hair follicle that contains multipotent stem cells.

### Example 4

### GLP-1 Induced Phosphorylation of ERK1/2 in Cultured Skin-Derived Cells

To determine whether GLP-1 receptors are actively coupled to signal transduction pathways in skin-derived cells, GLP-1 was added to the cells and examined for the activation of cell signaling. Three signaling pathways were investigated: Cyclic AMP, calcium influx, and mitogen activated protein kinase phosphorylation (MAPK).

Since GLP-1Rs are classically recognized to couple to Gs and adenylyl cyclase to generate cAMP cell extracts were assayed for changes in levels of cAMP after their incubation with GLP-1 for 5-30 min. No changes in cAMP levels were observed (data not shown).

Further, a cAMP luciferase reporter plasmid transfected to the cultured skin-derived cells failed to activate in response to GLP-1 (data not shown).

The cells were next loaded with the Ca2+indicator fora-2, and changes in membrane potential and current were monitored using the perforated patch clamp technique. No membrane current was detected in response to GLP-1 (data not shown).

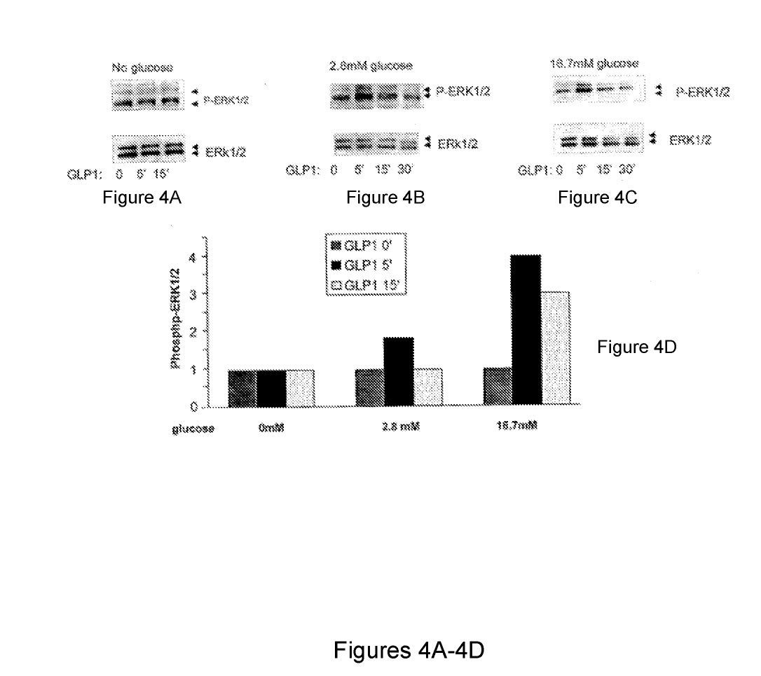

The cells were next assayed for the activation of the components of the MAPK signaling pathway, P42/44 MAPK (ERK1/2) by Western immunoblotting using antisera to ERK1/2 and to phosphorylated ERK2 (P-ERK2) with some cross-reactivity with phosphorylated ERK1 (P-ERK1) (FIGS. 4A-4D). Primary skin cell cultures were established from C57B1/6 mice. After preincubation in serum-free medium for one hour, the cultures were stimulated with 10 nM GLP-1 for the indicated amount of time (0, 5, 15 or 30 minutes).

FIG. 4A depicts a Western blot showing ERK phosphorylation in response to GLP-1 stimulation of primary skin cell cultures in the presence of no glucose. The upper blot depicts phosphorylated ERK (42 and 44 kd, arrows) detected by blotting with a phospho ERK—specific antiserum. The lower blot depicts total ERK1/2 protein.

FIG. 4B depicts a Western blot showing ERK phosphorylation in response to GLP-1 stimulation of primary skin cell cultures in the presence of low glucose concentration (2.8 mM glucose). The upper blot depicts phosphorylated ERK (42 and 44kd, arrows) detected by blotting with a phospho ERK—specific antiserum. The lower blot depicts total ERK1/2 protein.

FIG. 4C depicts a Western blot showing ERK phosphorylation in response to GLP-1 stimulation of primary skin cell cultures in the presence of a high glucose concentration (16.7 mM glucose). The upper blot depicts phosphorylated ERK (42 and 44 kd, arrows) detected by blotting with a phospho ERK—specific antiserum. The lower blot depicts total ERK1/2 protein.

FIG. 4D depicts a semi-quantitative densitometric measurement of relative phosphorylation responses shown in FIG. 4A-4C. Phosphorylated forms of ERK increase in abundance in both high and low glucose conditions by 5 minutes and to come down again by 15 minutes.

Addition of GLP-1 to the cells elicited a prompt phosphorylation of ERK1/2 after 5 minutes followed by defervescence of phosphorylation at 15 and rephosphorylation at 30 min, showing characteristic burst-decay kinetics of phosphorylation typical of active protein kinase/phosphatase signaling Moreover, the phosphorylation of ERK1/2 in response to GLP-1 was glucose-dependent, similar to the glucose dependency of GLP-1 insulinotropic actions on pancreatic beta cells.

Hence, GLP-1 activates MAPK/ERK signaling in cultured skin-derived cells. GLP-1 elicits a prompt activation of the MAPK (ERK1/2) signaling pathway in the absence of cAMP signaling. This example also demonstrates that the activation of ERK1/2 by GLP-1 in the cultured skin cells was glucose-dependent.

Hence, the inventors have discovered that the activation of the MAPK (ERK1/2) signaling pathway by GLP-1 in cultured skin cells indicates that the GLP-1 and GLP-1R expression seen in skin and hair follicles functions to promote cell growth and differentiation; and that GLP-1 is a paracrine/autocrine regulator of skin development and folliculogenesis.

### Example 5

Differentiation of Cultured Skin-Derived Cells into Insulin C-Peptide Producing Cells

Mouse skin was minced and strained, and the resulting cells were used to establish primary cultures of mouse skin-derived cells. Culture conditions for mouse skin-derived cells were switched from growth in expansion media containing high serum (10%), bFGF, and EGF, to differentiation media containing low serum (2%), supplement B-27, pyruvate, and GLP-1 (10 nM). The cells were maintained in differentiation media for 30 days. Following differentiation, cells were fixed with 4% paraformaldehyde and immunostained with an antiserum specific for insulin C-peptide, a marker of the expression of the insulin gene. Immunoreactivity was detected with a Cy-3-conjugated secondary antibody. Many of the cells showed cytoplasmic staining of C-peptide.

Immunostaining was performed in primary mouse skin cultures treated with GLP-1 or using (a) αC-peptide or (b) normal rabbit serum (NRS) as a control (data not shown). Cytoplasmic staining with C-peptide antiserum is seen in viable-appearing cells as well as in condensed, possibly apoptotic nuclei, whereas no cytoplasmic staining is seen with NRS.

Hence, the inventors have discovered that cultured skin-derived cells can dedifferentiate and redifferentiate into insulin-expressing cells (as detected by the presence C-peptide) upon exposure of the cells to GLP-1. C-peptide refers to a peptide molecule which is derived (cleaved from) from proinsulin its processing to mature insulin. C-peptide is produced at a 1:1 molar ratio with insulin, and is only produced in the setting of insulin production. Commercially-produced insulin does not contain C-peptide, so in systems where exogenous (commercially produced) insulin is present, such as cell culture in the presence of supplement B-27, the presence of C-peptide constitutes evidence of insulin production by the cells, whereas the presence of insulin reflects what has been added to the culture medium as well as what may have been produced by the cells.

### Example 6

### Nestin Expression in Skin and Cultured Skin-Derived Cells

Because the intermediate filament protein nestin, initially identified as a marker of neural stem cells, is believed to represent a characteristic marker of multi-lineage progenitor cells (reviewed in (13)), and is expressed in multipotent dermis-derived cells (Toma, J. G. et al, Nat. Cell Biol. 3:778-84 (2001)) and in hair follicle sheath progenitor cells (Li, L. et al., Proc. Natl. Acad. Sci. U.S.A. 100:9958-9961 (2003)), we examined mouse skin and cultured skin-derived cells for the expression of nestin.

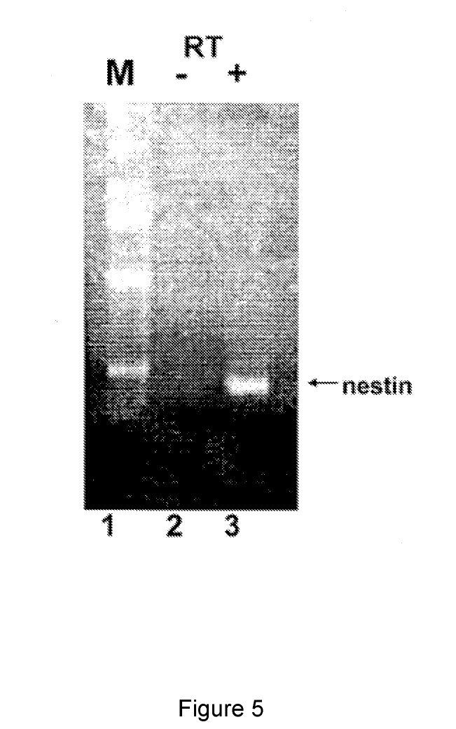

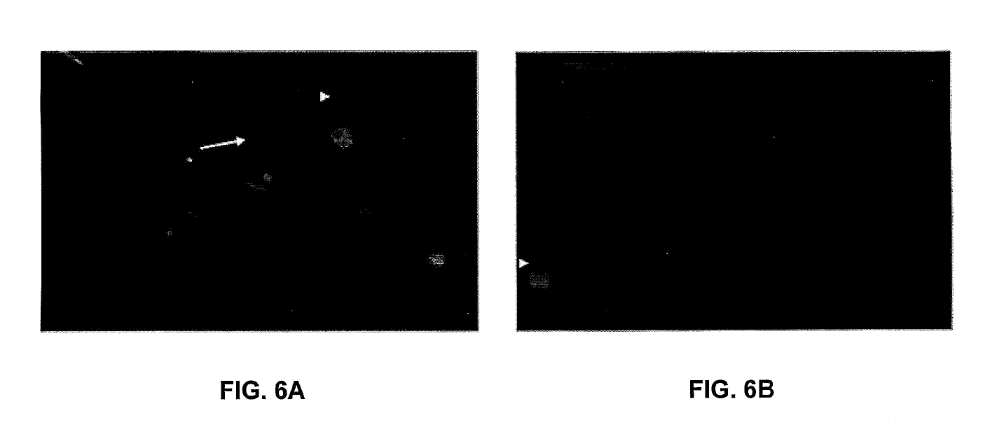

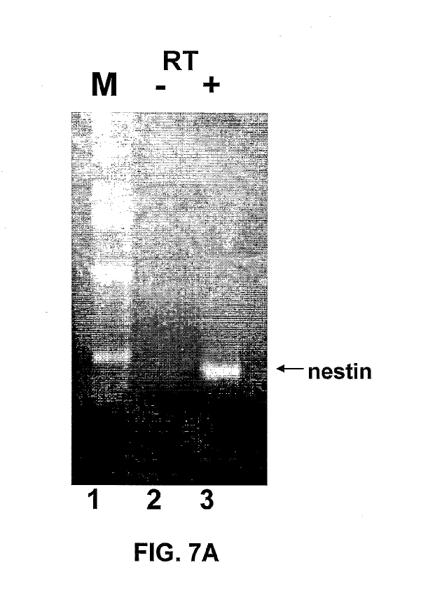

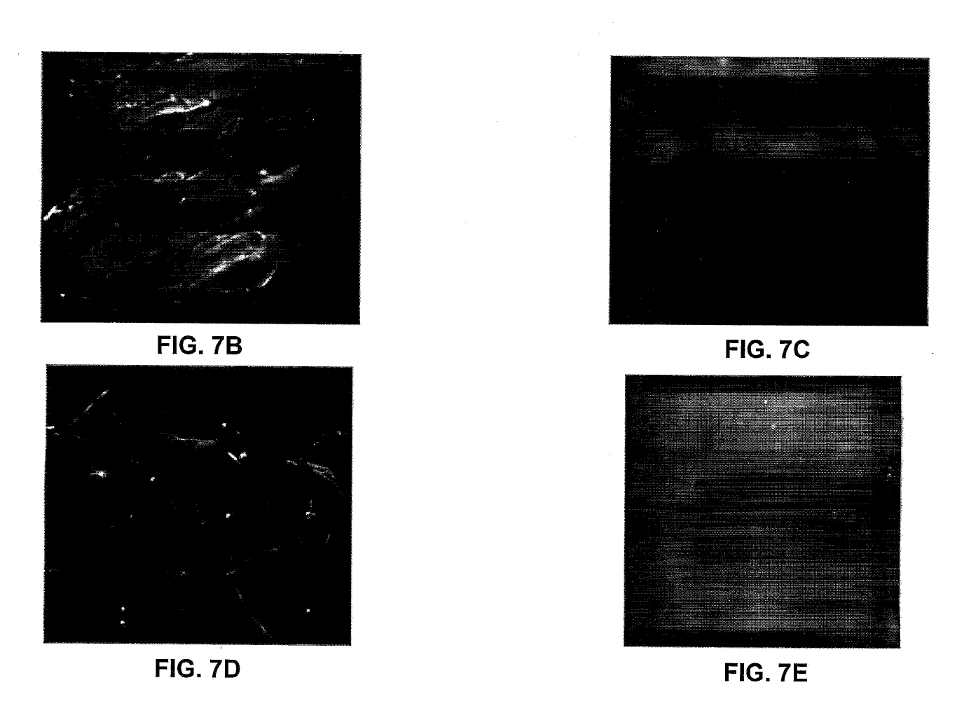

Expression of messenger RNA for nestin was readily detected by RT-PCR in extracts of cultured cells (FIG. 5) and skin (not shown). The products generated by RTPCR were cloned and sequenced, showing identity to the sequence of mouse nestin (NCBI No . gi15011850/NM016701.2). Nestin protein was also detected by immunochemistry in localized regions of hair follicles in skin (FIG. 5) and in the majority of skin-derived cells in culture (data not shown).

FIG. 5 depicts RT-PCR of nestin mRNA in cultured mouse skin-derived cells. Lane 1: Marker (M). Lane 2: without reverse transcriptase. Lane 3: RT-PCR product of expected size (arrow). The product was cloned and sequenced to verify its identity.

Immunostaining was performed using mouse monoclonal antiserum to nestin or control serum.

Hence, cultured skin cells express the neural stem cell protein, nestin, a marker of dedifferentiated epithelial cells (epithelial to mesenchymal transition, EMT). Thus, the inventors have discovered via culturing skin-derived cells the ability to propagate indigenous multipotent stem cells resident in the skin; and that GLP-1 causes endothelial to mesenchymal transition, or cell dedifferentiation.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventors, and thus, are not intended to limit the present invention and the appended claims in any way.

All publications mentioned above are hereby incorporated in their entirety by reference.

Having thus described embodiments of the invention, it should be apparent to those skilled in the art that certain advantages of the described method and apparatus have been achieved. It should also be appreciated that various modifications, adaptations, and alternative embodiments thereof may be made within the scope and spirit of the present invention. The invention is further defined by the following claims.

What is claimed is:

1. A method of stimulating hair growth comprising administering to a mammal GLP-1, a GLP-1 fragment or a GLP-1 derivative.

2. The method of claim 1, wherein the GLP-1, GLP-1 fragment or GLP-1 derivative is applied topically.

3. The method of claim 1, wherein the GLP-1, GLP-1 or GLP-1 derivative is administered via microneedle injections, jet injections, pressure waves, or ultrasound.

4. The method of claim 1, wherein said mammal is a human.

5. The method of claim 1, comprising administering GLP-1, GLP1(7-37) or GLP-1(7-36) amide.


SEQUENCE LISTING (113 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 37 aa, Gallus gallus
HSEFERHAEGTYTSDITSYLEGQAAKEFIAWLVNGRG

SEQ ID NO: 3 - protein, 31 aa, Gallus gallus
HAEGTYTSDITSYLEGQAAKEFIAWLVNGRG

SEQ ID NO: 4 - protein, 27 aa, Gallus gallus
HSEGTFTSDFTRYLDKMKAKDFVHWLI

SEQ ID NO: 5 - protein, 37 aa, Agkistrodon piscivorus
HAEYERHADGTYTSDISSYLEGQAAKEFIAWLVNGRG

SEQ ID NO: 6 - protein, 31 aa, Agkistrodon piscivorus
HADGTYTSDISSYLEGQAAKEFIAWLVNGRG

SEQ ID NO: 7 - protein, 37 aa, Heloderma suspectum
HAEYERHADGRYTSDISSYLEGQAAKEFIAWLVNGRG

SEQ ID NO: 8 - protein, 31 aa, Heloderma suspectum
HADGRYTSDISSYLEGQAAKEFIAWLVNGRG

SEQ ID NO: 9 - protein, 29 aa, Heloderma suspectum
HGEGTFTSDLSKQMEEEAVRLFIEWLKNG

SEQ ID NO: 10 - protein, 34 aa, Heloderma suspectum
SKIKRHGEGTFTSDLSKQMEEEAVRLFIEWLKNG

SEQ ID NO: 11 - protein, 29 aa, Heloderma horridum
HSDGTFTSDLSKQMEEEAVRLFIEWLKNG

SEQ ID NO: 12 - protein, 34 aa, Heloderma horridum
SKIKRHSDGTFTSDLSKQMEEEAVRLFIEWLKNG

SEQ ID NO: 13 - protein, 37 aa, Neoceratodus forsteri
HSEIERHAEGTYTSDISSYLEGQAANEFVRWLLKGRG

SEQ ID NO: 14 - protein, 31 aa, Neoceratodus forsteri
HAEGTYTSDISSYLEGQAANEFVRWLLKGRG

SEQ ID NO: 15 - protein, 37 aa, Protopterus dolloi
HNEIERHAEGTYTSDISSYLEGQAVNEFIRWLMKGRG

SEQ ID NO: 16 - protein, 31 aa, Protopterus dolloi
HAEGTYTSDISSYLEGQAVNEFIRWLMKGRG

SEQ ID NO: 17 - protein, 34 aa, Xenopus laevis
EFERHAEGTYTNDVTEYLEEKAAKEFIEWLIKGK

SEQ ID NO: 18 - protein, 33 aa, Xenopus laevis
YSRHAEGTFTNDMTNYLEEKAAKEFVGWLIKGR

SEQ ID NO: 19 - protein, 33 aa, Xenopus laevis
DYERHAEGTFTSDVTQQLDEKAAKEFIDWLING

SEQ ID NO: 20 - protein, 30 aa, Xenopus laevis
HAEGTFTNDMTNYLEEKAAKEFVGWLIKGR

SEQ ID NO: 21 - protein, 30 aa, Xenopus laevis
HAEGTYTNDVTEYLEEKAAKEFIEWLIKGK

SEQ ID NO: 22 - protein, 29 aa, Xenopus laevis
HAEGTFTSDVTQQLDEKAAKEFIDWLING

SEQ ID NO: 23 - protein, 27 aa, Xenopus laevis
HSQGTFTSDYSKYLDSRRAQDFVQWLM

SEQ ID NO: 24 - protein, 27 aa, Xenopus laevis
HADGSFTNDINKVLDIIAAQEFLDWVI

SEQ ID NO: 25 - protein, 34 aa, Lophius americanus
FRRHADGTYTSDVSSYLQDQAAKDFVSWLKAGRG

SEQ ID NO: 26 - protein, 34 aa, Tetraodon nigroviridis
FRRHADGTYTSDVSAYLQDQAAKEFVSWLKTGRG

SEQ ID NO: 27 - protein, 31 aa, Tetraodon nigroviridis
HADGTYTSDVSAYLQDQAAKEFVSWLKTGRG

SEQ ID NO: 28 - protein, 26 aa, Tetraodon nigroviridis
HSEGTFSNDYSKYLETRRAQDFVQWL

SEQ ID NO: 29 - protein, 30 aa, Tetraodon nigroviridis
HADGTFTSDVSSYLKDQAIKDFVARLKAGQ

SEQ ID NO: 30 - protein, 34 aa, Sebastes caurinus
FRRHADGTYTSDVSSYLQDQAAKEFVYWLKTGRG

SEQ ID NO: 31 - protein, 31 aa, Sebastes caurinus
HADGTYTSDVSSYLQDQAAKEFVYWLKTGRG

SEQ ID NO: 32 - protein, 26 aa, Sebastes caurinus
HSEGTFSNDYSKYLETRRAQDFVQWL

SEQ ID NO: 33 - protein, 30 aa, Sebastes caurinus
HADGTFTSDVSSYLKDQAIKDFVNRLKSGQ

SEQ ID NO: 34 - protein, 30 aa, Lophius americanus
HADGTFTSDVSSYLKDQAIKDFVDRLKAGQ

SEQ ID NO: 35 - protein, 30 aa, Lophius americanus
HSEGTFSNDYSKYLEDRKAQEFVRWLMNNK

SEQ ID NO: 36 - protein, 32 aa, Danio rerio
RHADGTYTSDVSSYLQDQAAKEFVSWLKTGRG

SEQ ID NO: 37 - protein, 31 aa, Danio rerio
HADGTYTSDVSSYLQDQAAKEFVSWLKTGRG

SEQ ID NO: 38 - protein, 30 aa, Danio rerio
HAEGTYTSDVSSYLQDQAAQSFVAWLKSGQ

SEQ ID NO: 39 - protein, 30 aa, Danio rerio
HSEGTFSNDYSKYLETRRAQDFVQWLMNAK

SEQ ID NO: 40 - protein, 30 aa, Danio rerio
HAEGTYTSDVSSYLQDQAAQRFVARLKSGQ

SEQ ID NO: 41 - protein, 34 aa, Hoplobatrachus rugulosus
QFERHAEGTYTNDVTQFLEEKAAKEFIDWLIKGK

SEQ ID NO: 42 - protein, 30 aa, Hoplobatrachus rugulosus
HAEGTFTSDMTSYLEEKAAKEFVDWLIKGR

SEQ ID NO: 43 - protein, 30 aa, Hoplobatrachus rugulosus
HAEGTYTNDVTQFLEEKAAKEFIDWLIKGK

SEQ ID NO: 44 - protein, 33 aa, Xenopus tropicalis; identical to SEQ ID NO: 18

SEQ ID NO: 45 - protein, 34 aa, Xenopus tropicalis
DVERHAEGTYTNDVTEYLEEKAAKEFIEWLINGK

SEQ ID NO: 46 - protein, 33 aa, Xenopus tropicalis
DYERHAEGTFTSDVTQHLDEKAAKEFIDWLING

SEQ ID NO: 47 - protein, 30 aa, Xenopus tropicalis; identical to SEQ ID NO: 20

SEQ ID NO: 48 - protein, 30 aa, Xenopus tropicalis
HAEGTYTNDVTEYLEEKAAKEFIEWLINGK

SEQ ID NO: 49 - protein, 29 aa, Xenopus tropicalis
HAEGTFTSDVTQHLDEKAAKEFIDWLING

SEQ ID NO: 50 - protein, 27 aa, Xenopus tropicalis
HSQGTFTSDYSKYLDSRRAQDFIQWLM

SEQ ID NO: 51 - protein, 32 aa, Ictalurus punctatus
RHADGTYTSDVSSYMQDQAAKEFVSWLKMGRG

SEQ ID NO: 52 - protein, 31 aa, Ictalurus punctatus
HADGTYTSDVSSYMQDQAAKEFVSWLKMGRG

SEQ ID NO: 53 - protein, 30 aa, Ictalurus punctatus
HSEGTFSNDYSKYLETRRAQDFIQWLMNSK

SEQ ID NO: 54 - protein, 31 aa, Amphiuma tridactylum
HADGTLTSDISSFLEKQATKEFIAWLVSGRG

SEQ ID NO: 55 - protein, 30 aa, Bullfrog
HADGTFTSDMSSYLEEKAAKEFVDWLIKGR

SEQ ID NO: 56 - protein, 31 aa, Lophius americanus
HADGTYTSDVSSYLQDQAAKDFVSWLKAGRG

SEQ ID NO: 57 - protein, 26 aa, Lophius americanus
HSEGTFSNDYSKYLETRRAQDFVQWL

SEQ ID NO: 58 - protein, 30 aa, Eel
HAEGTYTSDVSSYLQDQAAKEFVSWLKTGR

SEQ ID NO: 59 - protein, 30 aa, Bufo marinus
HAEGTFTSDMTSFLEEKAAKEFVDWLIKGR

SEQ ID NO: 60 - protein, 29 aa, Bufo marinus
HAEGTYTNDVTQFLEEKAAKEFIDWLLKG

SEQ ID NO: 61 - protein, 27 aa, Bufo marinus
HADGSFTSDFNKALDIKAAQEFLDWII

SEQ ID NO: 62 - protein, 30 aa, Oncorhynchus mykiss
HADGTYTSDVSTYLQDQAAKDFVSWLKSGR

SEQ ID NO: 63 - protein, 30 aa, Oncorhynchus tshawytscha; identical to SEQ ID NO: 62

SEQ ID NO: 64 - protein, 30 aa, Channel catfish
HADGTYTSDVSSYLQDQAAKDFITWLKSGQ

SEQ ID NO: 65 - protein, 27 aa, Channel catfish
HSEGTFSNDYSKYLETRRAQDFVQWLM

SEQ ID NO: 66 - protein, 30 aa, Ictalurus punctatus; identical to SEQ ID NO: 64

SEQ ID NO: 67 - protein, 29 aa, Alligator gar
HADGTYTSDVSSYLQDQAAKKFVTWLKQG

SEQ ID NO: 68 - protein, 27 aa, Alligator gar
HSQGTFTNDYSKYLDTRRAQDFVQWLM

SEQ ID NO: 69 - protein, 28 aa, Tilapia nilotica
GTYTSDVSSYLQDQAAKEFVSWLKTGRG

SEQ ID NO: 70 - protein, 29 aa, Rainbow trout
HADGTYTSDVSTYLQDQAAKDFVSWLKSG

SEQ ID NO: 71 - protein, 30 aa, Coho salmon
HADGTYTSNVSTYLQDQAAKDFVSWLKSGR

SEQ ID NO: 72 - protein, 27 aa, Scyliorhinus canicula
HSEGTFTSDYSKYMDNRRAKDFVQWLM

SEQ ID NO: 73 - protein, 27 aa, Smaller spotted catshark
HSEGTFTSDYSKYMDNRRAKDFVQWLM

SEQ ID NO: 74 - protein, 30 aa, Polyodon spathula
HADGTYTSDASSFLQEQAARDFISWLKKGQ

SEQ ID NO: 75 - protein, 30 aa, Polyodon spathula
HSQGMFTNDYSKYLEEKSAKEFVEWLKNGK

SEQ ID NO: 76 - protein, 30 aa, Squalus acanthias
HTEGSYRDISSYLEAKAAKDFINWLIKGRG

SEQ ID NO: 77 - protein, 30 aa, Shorthorn sculpin
HADGTFTSDVSSYLNDQAIKDFVAKLKSGK

SEQ ID NO: 78 - protein, 26 aa, Shorthorn sculpin
HSEGTFSNDYSKYLETRRAQDFVQWL

SEQ ID NO: 79 - protein, 28 aa, Petromyzon marinus
HADGTFTNDMTSYLDAKAARDFVSWLAR

SEQ ID NO: 80 - protein, 30 aa, Petromyzon marinus
HSEGTFTSDYSKYLENKQAKDFVRWLMNAK

SEQ ID NO: 81 - protein, 27 aa, Petromyzon marinus
HSEGTFTSDYSKYLENKQAKDFVRWLM

SEQ ID NO: 82 - protein, 27 aa, Marbled electric ray
HSEGTFTSDYSKYLDNRRAKDFVQWLM

SEQ ID NO: 83 - protein, 29 aa, Hydrolagus colliei
EEDYPNDFSSYLEAKAAKDFIDWLIKGQG

SEQ ID NO: 84 - protein, 27 aa, Callorhynchus milii
HSEGTFSSDYSKYLDSRRAKDFVQWLM

SEQ ID NO: 85 - protein, 31 aa, Artificial sequence; identical to SEQ ID NO: 37
  GLP-1 derivative

SEQ ID NO: 86 - protein, 30 aa, Artificial sequence
  GLP-1 derivative
HADGTYTSDVSSYLQDQAAKKFVTWLKQGQ

SEQ ID NO: 87 - protein, 30 aa, Artificial sequence; identical to SEQ ID NO: 71
  GLP-1 derivative

SEQ ID NO: 88 - protein, 30 aa, Artificial sequence
  GLP-1 derivative
HADGTYTSDVSSYLQEQAAKDFITWLKSGQ

SEQ ID NO: 89 - protein, 30 aa, Artificial sequence; identical to SEQ ID NO: 77
  GLP-1 derivative

SEQ ID NO: 90 - protein, 30 aa, Artificial sequence
  GLP-1 derivative
HAEGTYTSDISSFLRDQAAQNFVAWLKSGQ

SEQ ID NO: 91 - protein, 30 aa, Artificial sequence; identical to SEQ ID NO: 55
  GLP-1 derivative

SEQ ID NO: 92 - protein, 27 aa, Artificial sequence
  GLP-1 derivative
HSEGTFSNDYSKYLETQRAQDFVQWLM

SEQ ID NO: 93 - protein, 27 aa, Artificial sequence
  GLP-1 derivative
HSEGTFSNDYSKYLETRRAQDFVQWLM

SEQ ID NO: 94 - protein, 27 aa, Artificial sequence
  GLP-1 derivative
HSQGTFTSDYSKYLDSKKAQEFVQWLM

SEQ ID NO: 95 - protein, 27 aa, Artificial sequence
  GLP-1 derivative
HSQGTFTSDYSKYLDNRRAQDFIQWLM

SEQ ID NO: 96 - protein, 26 aa, Artificial sequence
  GLP-1 derivative
HSEGTFSNDYSKYLETRRAQDFVQWL

SEQ ID NO: 97 - protein, 39 aa, Artificial sequence
  GLP-1 derivative
HSDGTFTSDLSKQMEEEAVRLFIEWLKNGGPSSGAPPPS

SEQ ID NO: 98 - protein, 39 aa, Artificial sequence
  GLP-1 derivative
HGEGTFTSDLSKQMEEEAVRLFIEWLKNGGPSSGAPPPS

SEQ ID NO: 99 - protein, 31 aa, Artificial sequence
  GLP-1 derivative
XXEGTFTSDVSSYLXGQAAKXFIAWLVKGRX

SEQ ID NO: 100 - DNA, 29 bp, Artificial sequence
  GLP-1 forward primer
atggccagcaccccaagcctcctgcgcct

SEQ ID NO: 101 - DNA, 29 bp, Artificial sequence
  GLP-1 reverse primer
actcttcacactccgacaggtccctccag

SEQ ID NO: 102 - DNA, 29 bp, Artificial sequence
  GLP-1 forward primer
ggtaccacggtgtccctctcagagacggt

SEQ ID NO: 103 - DNA, 29 bp, Artificial sequence
  GLP-1 reverse primer
gggcagccgtgctatacatccacttgagg

SEQ ID NO: 104 - DNA, 29 bp, Artificial sequence
  GLP-1 forward primer
cctgaggaacagctcctgtccctgtacat

SEQ ID NO: 105 - DNA, 29 bp, Artificial sequence
  GLP-1 reverse primer
gtgaacagcttgatgaagcgtagggttcc

SEQ ID NO: 106 - DNA, 29 bp, Artificial sequence
  GLP-1 forward primer
tcggtgttctcggagcagcgcatcttcaa

SEQ ID NO: 107 - DNA, 29 bp, Artificial sequence
  GLP-1 reverse primer
ctcattgttgacaaagcagtacaggatag

SEQ ID NO: 108 - DNA, 22 bp, Artificial sequence
  Proglucagon forward primer
agaagaagtcgccattgctgag

SEQ ID NO: 109 - DNA, 26 bp, Artificial sequence
  Proglucagon reverse primer
gttctctttccaggtttaccactacg

SEQ ID NO: 110 - DNA, 23 bp, Artificial sequence
  Proglucagon forward primer
agaagcgcatgaggaccctgatg

SEQ ID NO: 111 - DNA, 25 bp, Artificial sequence
  Proglucagon reverse primer
acgagatgttgtgaagatggttgtg

SEQ ID NO: 112 - DNA, 21 bp, Artificial sequence
  Nestin forward primer
agccgaggaccagatacttga

SEQ ID NO: 113 - DNA, 24 bp, Artificial sequence
  Nestin reverse primer
gactcctgactttccttttctacc